SEQUENCE LISTING (60 sequences)

SEQ ID NO: 1 - DNA, 4304 bp, Homo sapiens
  CDS (459)..(2033)
gcgagaactcatcctgtagtcaccagatggagtcccaaacagccaagcagatgtaaggcctgtgctgtggctctgaggccctgaatacagaagggtcactttcttagtggccaaagagcagttgttgacattgatgtctaattattgaacacgaccagtcattttactgagctgcggtgaggaaacactgaccatagaagatcaagccaaatgagggattgcaaatttcctgattcttttgaattaggattccagatgggggcctcatttctacagcccccaacattcctatagccgttatcactgccatcaccactgccaccagcatcttcttgcagattccacccctgctccccagagacttcctgctttgaaagtgagcagaaaggaagctctcagaaaaatctctagtggtggctgccgtcgctccagacaatcggaatcctgccttcaccaccatgggctggctttttctaaaggttttgttggcgggagtgagtttctcaggatttctttatcctcttgtggatttttgcatcagtgggaaaacaagaggacagaagccaaactttgtgattattttggccgatgacatggggtggggtgacctgggagcaaactgggcagaaacaaaggacactgccaaccttgataagatggcttcggagggaatgaggtttgtggatttccatgcagctgcctccacctgctcaccctcccgggcttccttgctcaccggccggcttggccttcgcaatggagtcacacgcaactttgcagtcacttctgtgggaggccttccgctcaacgagaccaccttggcagaggtgctgcagcaggcgggttacgtcactgggataataggcaaatggcatcttggacaccacggctcttatcaccccaacttccgtggttttgattactactttggaatcccatatagccatgatatgggctgtactgatactccaggctacaaccaccctccttgtccagcgtgtccacagggtgatggaccatcaaggaaccttcaaagagactgttacactgacgtggccctccctctttatgaaaacctcaacattgtggagcagccggtgaacttgagcagccttgcccagaagtatgctgagaaagcaacccagttcatccagcgtgcaagcaccagcgggaggcccttcctgctctatgtggctctggcccacatgcacgtgcccttacctgtgactcaactaccagcagcgccacggggcagaagcctgtatggtgcagggctctgggagatggacagtctggtgggccagatcaaggacaaagttgaccacacagtgaaggaaaacacattcctctggtttacaggagacaatggcccgtgggctcagaagtgtgagctagcgggcagtgtgggtcccttcactggattttggcaaactcgtcaagggggaagtccagccaagcagacgacctgggaaggagggcaccgggtcccagcactggcttactggcctggcagagttccagttaatgtcaccagcactgccttgttaagcgtgctggacatttttccaactgtggtagccctggcccaggccagcttacctcaaggacggcgctttgatggtgtggacgtctccgaggtgctctttggccggtcacagcctgggcacagggtgctgttccaccccaacagcggggcagctggagagtttggagccctgcagactgtccgcctggagcgttacaaggccttctacattaccggtggagccagggcgtgtgatgggagcacggggcctgagctgcagcataagtttcctctgattttcaacctggaagacgataccgcagaagctgtgcccctagaaagaggtggtgcggagtaccaggctgtgctgcccgaggtcagaaaggttcttgcagacgtcctccaagacattgccaacgacaacatctccagcccagattacactcaggacccttcagtaactccctgctgtaatccctaccaaattgcctgccgctgtcaagccgcataacagaccaatttttattccacgaggaggagtacctggaaattaggcaagtttgcttccaaatttcatttttaccctctttacaaacacacgctttagtttagtcttggagtttagttttggagttagccttgcatatcccttctgtatcctgtccctcctccacgccgacccgagagcagctgagctgcgctggctctgggcagggagtgtgccttaatgggaagcacacgggctttggagtcaggcacaggtgccagctccagcttttgaacttgggcaattgtttaacctaacctgcaagttgattttgagggttaaataaaggcatacatgaaaatgcctggcaaattacctgacacagagcagacattcaatacattttagtttccttgtttctctggttcccagtttctctggtcattttggtgtaaatccattctaattagtatttagggcagagcttctctctcttttctctttttttccttccacaaaccagtgtactcactggtctccatctttaatatgcaaacaaatcacctgggatcttgtgagaatccggattctgtctcagtagggctcgagtagatcctgaaatcctacatttctatcaaacaatgccttgaggagcacagatttagaccaaagttaggtcgttttccagatctcagagcagacgagtccatggataagtctgtggcccaatccccttcctctccttttaagggtgaaatgactgcatttaaaagaagttaaagagttcctcctgtcccctataaccacaaggaaacaaaaaaatatataaaaacctcaaaaatgcattgccatgattttattattagtgtccaaaatgggactcccaagtaataaatgatttattccagccacagccaaaaaagactttgcctggctaaaagagtctctctctaagtatgtaatatacaagaaatacaattcaaagagatgttcctataagtacattttttacacggcatatatttaaaaaggaggccccttttaatataaaattccggttatataccaatatggttaattagcatttacactatagtttgaacgtattttaaatagcatgatgtgtatacaatgtctcccgcgcccattggcaaccagggtcgtgggaagcttggtgaggagttaaccaggtcctgtggtttaagcagtggagcacccgggattcctgcccccctttctgctcacacaattgcactccattcttccgccttccttgttttctccaaaaccacctgataggggggatgtcctgatttctgaggtgtgcttctcatcatgactgcttcgttttgcccttctgatttccacggcacaagattatctaccaaaatcaaaacagaatggccttactcttctcaggaagaggctggtaggcaggtgcattatcaacaggtctgtgcccatgcagagtgagcagggagaggctgggcactgtggaatttttctgtctgaactcgctcatggccacagaatggtcacccagcttatttaggtgtagacaagtatgacacagttctagaaaatactgactataaaaatgtctctgtgtgtgtgtgtatgtatttatatgtatatgtatatatttttaaaaggctcatcttacttgtaaacatggactgctcaatcactattaaaaagtcagtttaggctgggcgcggtggctcacgcctgtagtcccagagctttgggaggctgaggtgggtggatcactgggtcaggagtttgagaccagcctggccaacatggtgaaaccccatcgctactaaaaaatacaaaaattagccgggcatggtggcgctcacctgtaatcccggctactcgggaggctgaggcaggagagaatcgcttgaaccggggaggtggaggctgcagtgagccgagatcgcaccactgcactccagcctgggtgatggagcaagactccatctcaaaaaaaaaaaagtcagtttaggctgggcgcagtggctcacacctgtagtcccagcactttaggaggctgaggggggtgatcacctgaggtcaggagtttgagaccagcctggccaacatggtgaaatcctgtctctgctaaaaatacaaaatttagctgggcatggtggcgtgcctgaaaccccagctacttgggaggctgaggcactagaatcgcttgagcctgggaggtggaggttgcagtgagtggagatcgcgccaacacattctagcctgagggacagagtgagactctatcatctc

SEQ ID NO: 2 - protein, 525 aa, Homo sapiens
MGWLFLKVLLAGVSFSGFLYPLVDFCISGKTRGQKPNFVIILADDMGWGDLGANWAETKDTANLDKMASEGMRFVDFHAAASTCSPSRASLLTGRLGLRNGVTRNFAVTSVGGLPLNETTLAEVLQQAGYVTGIIGKWHLGHHGSYHPNFRGFDYYFGIPYSHDMGCTDTPGYNHPPCPACPQGDGPSRNLQRDCYTDVALPLYENLNIVEQPVNLSSLAQKYAEKATQFIQRASTSGRPFLLYVALAHMHVPLPVTQLPAAPRGRSLYGAGLWEMDSLVGQIKDKVDHTVKENTFLWFTGDNGPWAQKCELAGSVGPFTGFWQTRQGGSPAKQTTWEGGHRVPALAYWPGRVPVNVTSTALLSVLDIFPTVVALAQASLPQGRRFDGVDVSEVLFGRSQPGHRVLFHPNSGAAGEFGALQTVRLERYKAFYITGGARACDGSTGPELQHKFPLIFNLEDDTAEAVPLERGGAEYQAVLPEVRKVLADVLQDIANDNISSPDYTQDPSVTPCCNPYQIACRCQAA

SEQ ID NO: 3 - DNA, 2258 bp, Homo sapiens
  CDS (20)..(1201)
ggcgttggctgctagagcgatgccgggccggagttgcgtcgccttagtcctcctggctgccgcgtcagctgtgccgtcgcagcacgcgccgccgtggacagaggactgcagaaaatcaacctatcctccttcaggaccaacgtacagaggtgcagttccatggtacaccataaatcttgacttaccaccctacaaaagatggcatgaattgatgcttgacaaggcaccaatgctaaaggttatagtgaattctctgaagaatatgataaatacattcgtgccaagtggaaaagttatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgccatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtgagcacacgtctggcctacagaatgcggcctctgagacatgaagacaccatctccatgtgaccgaacactgcagctgtctgaccttccaaagactaagactcgcggcaggttctctttgagtcaatagcttgtcttcgtccatctgttgacaaatgacagactttttttttttccccctatcagttgatttttcttatttatagataacttctttaggggaagtaaaacagtcatctagaattcactgagttttgtttcactttgacatttggggatctggtgggcagtcgaaccatggtgaactccacctccgtgaataaatggagattcagcgtgggtgttgaatccagcacgtctgtgtgagtaacgggacagtaaacactccacattcttcagtttttcacttctacctacatatttgtatgtttttctgtataacagccttttccttctggttctaactgctgttaaaattaatatatcattatctttgctgttattgacagcgatataattttattacatatgattagagggatgagacagacattcacctgtatatttcttttaatgggcacaaaattggtgcctttgcctctaaatagcactttttcggggtcaagaagtaatcagatgcaaagcaatcgtttatacaataattgaagcgcacctttcaataccactccagtacctaaggaagtgctactaaactgcatccacgtctgtatagtaataacagtcaagctggaatcgaggaccaattaattccaatggcacagagtagcattcatgtaataaacaggtttttagtttgttcttcagattgatagggagttttaaagaaattttagtagttactaaaattatgttactgtatttttcagaaatccaactgcttatgaaaagtactaatagaacttgttaacctttctaaccttcacgattaactgtgaaatgtacgtcatttgtgcaagaccgtttgtccacttcattttgtataatcacagttgtgttcctgacactcaataaacagtcattggaaagagaaaaaaaaa

SEQ ID NO: 4 - protein, 394 aa, Homo sapiens [sequence omitted]

SEQ ID NO: 5 - DNA, 1336 bp, Homo sapiens
  CDS (474)..(1241)
agcacagtcccgcggacggctgagcgtgtggctgcaggagcttctgtgggagtacggtcatgagccttttgctgtactatgccctccctgccctgggcagctatgccatgctctccatcttcttcctgcgccggcctcatctgctgcacacgcccagggctcccaccttccgcatccgcctgggggcccaccgaggaggatctggagagctgctggagaacaccatggaggccatggagaagtgagtgtatctgcccctgcccaggctgccatgtgagggtgtgcaggtcgtggactgcatcacagcaagggacgccattcacttcctagctgggtgactggcgccccctggaggtggcctgcacatcccgccacgaactccgctctcacattcccggccacctcccagccaccatgcccctcatcccagtcccctgcccctgccccccgctgaccttcacccccacagctccatggcccagcgctcggacctcctggagctcgactgtcagctgacacgggacagagtggtggtggtgtcacatgatgagaacctgtgccgccagtcgggcctaaacagggatgtgggcagcctggacttcgaggacctgcccctctacaaggagaagctggaggtttacttctctccaggccactttgctcacgggtcagaccggcgcatggttcgtctggaggacctgttccagaggtttccaaggacacccatgagcgtagagatcaaagggaagaacgaagagctcatccgtgagatagcaggcttggtgagacgctatgaccgtaatgaaatcaccatctgggcctcggagaagagctcggtcatgaagaaatgcaaggctgccaaccccgagatgcccctgtccttcacaataagccgaggattctgggtgctgctttcctactacctggggctgctgcccttcatcccaatccctgagaagttcttcttctgcttcctgcccaacatcatcaacaggacctatttcccattttcctgctcttgcctgaaccagttattggctgtggtttcgaaatggctgatcatgaggaagagtctgatccgacacttggaggagcgaggggtgcaggtggtcttttggtgccttaatgaagagtcggattttgaagcagccttcagcgtgggagccactggcgtcataacggattatcccacagccctgcggcactacctggacaaccatggaccagctgcccggacctcctaagtccagaagcctcgaggtcttctgtttctcttcctgaaaaataaatatttgcctttcgatcaaaaaaaaaaaaaaaaaaaaaaaaaaaaaaa

SEQ ID NO: 6 - protein, 256 aa, Homo sapiens
MAQRSDLLELDCQLTRDRVVVVSHDENLCRQSGLNRDVGSLDFEDLPLYKEKLEVYFSPGHFAHGSDRRMVRLEDLFQRFPRTPMSVEIKGKNEELIREIAGLVRRYDRNEITIWASEKSSVMKKCKAANPEMPLSFTISRGFWVLLSYYLGLLPFIPIPEKFFFCFLPNIINRTYFPFSCSCLNQLLAVVSKWLIMRKSLIRHLEERGVQVVFWCLNEESDFEAAFSVGATGVITDYPTALRHYLDNHGPAARTS

SEQ ID NO: 7 - DNA, 2631 bp, Homo sapiens
  CDS (19)..(2214)
gagagcactgcagcagcaatgacggagggcacgtgtctgcggcgccgagggggcccctacaagaccgagcccgccaccgacctcggccgctggcgactcaactgcgagaggggccggcagacgtggacctacctgcaggacgagcgcgccggccgcgagcagaccggcctggaagcctacgccctggggctggacaccaagaattactttaaggacttgcccaaagcccacaccgcctttgagggggctctgaacgggatgacattttacgtggggctgcaggctgaggatgggcactggacgggtgattatggtggcccacttttcctcctgccaggcctcctgatcacttgccacgtggcacgcatccctctgccagccggatacagagaagagattgtgcggtacctgcggtcagtgcagctccctgacggtggctggggcctgcacattgaggataagtccaccgtgtttgggactgcgctcaactatgtgtctctcagaattctgggtgttgggcctgacgatcctgacctggtacgagcccggaacattcttcacaagaaaggtggtgctgtggccatcccctcctgggggaagttctggctggctgtcctgaatgtttacagctgggaaggcctcaataccctgttcccagagatgtggctgtttcctgactgggcaccggcacacccctccacactctggtgccactgccggcaggtgtacctgcccatgagctactgctacgccgttcggctgagtgccgcggaagacccgctggtccagagcctccgccaggagctctatgtggaggacttcgccagcattgactggctggcgcagaggaacaacgtggcccccgacgagctgtacacgccgcacagctggctgctccgcgtggtatatgcgctcctcaacctgtatgagcaccaccacagtgcccacctgcggcagcgggccgtgcagaagctgtatgaacacattgtggccgacgaccgattcaccaagagcatcagcatcggcccgatctcgaaaaccatcaacatgcttgtgcgctggtatgtggacgggcccgcctccactgccttccaggagcatgtctccagaatcccggactatctctggatgggccttgacggcatgaaaatgcagggcaccaacggctcacagatctgggacaccgcattcgccatccaggctctgcttgaggcgggcgggcaccacaggcccgagttttcgtcctgcctgcagaaggctcatgagttcctgaggctctcacaggtcccagataaccctcccgactaccagaagtactaccgccagatgcgcaagggtggcttctccttcagtacgctggactgcggctggatcgtttctgactgcacggctgaggccttgaaggctgtgctgctcctgcaggagaagtgtccccatgtcaccgagcacatccccagagaacggctctgcgatgctgtggctgtgctgctgaacatgagaaatccagatggagggttcgccacctatgagaccaagcgtggggggcacttgctggagctgctgaacccctcggaggtcttcggggacatcatgattgactacacctatgtggagtgcacctcagccgtgatgcaggcgcttaagtatttccacaagcgtttcccggagcacagggcagcggagatccgggagaccctcacgcagggcttagagttctgtcggcggcagcagagggccgatggctcctgggaaggctcctggggagtttgcttcacctacggcacctggtttggcctggaggccttcgcctgtatggggcagacctaccgagatgggactgcctgtgcagaggtctcccgggcctgtgacttcctgctgtcccggcagatggcagacggaggctggggggaggactttgagtcctgcgaggagcggcgttatttgcagagtgcccagtcccagatccataacacatgctgggccatgatggggctgatggccgttcggcatcctgacatcgaggcccaggagagaggagtccggtgtctacttgagaaacagctccccaatggcgactggccgcaggaaaacattgctggggtcttcaacaagtcctgtgccatctcctacacgagctacaggaacatcttccccatctgggccctcggccgcttctcccagctgtaccctgagagagcccttgctggccacccctgagaacatgcctacctgctgggtgccgtctgtgcgttccatggccttcaagtcacaggacgcagcgattccctgccctcttcggtgttattacacaggcaggacttcagtgtcagtatccctgccttcagtcttctttagaaatcacatctgtgttcaatccattgtttagagggagtgtatttttcctgttccacgaagaggactttttgttcacaattggatcacaatgcagaggagtctgttcctcccccgtcggcttctcggtgctgggagggtgacctgtcccagatgactcatcaccctgacatgctcttgacaaaggacaccaccaagaggagatggcagctgtaccggtgcagcctctgtctgagggggatatttgcctcagtgtgattaaaaatcagtcatgaaaga

SEQ ID NO: 8 - protein, 732 aa, Homo sapiens
MTEGTCLRRRGGPYKTEPATDLGRWRLNCERGRQTWTYLQDERAGREQTGLEAYALGLDTKNYFKDLPKAHTAFEGALNGMTFYVGLQAEDGHWTGDYGGPLFLLPGLLITCHVARIPLPAGYREEIVRYLRSVQLPDGGWGLHIEDKSTVFGTALNYVSLRILGVGPDDPDLVRARNILHKKGGAVAIPSWGKFWLAVLNVYSWEGLNTLFPEMWLFPDWAPAHPSTLWCHCRQVYLPMSYCYAVRLSAAEDPLVQSLRQELYVEDFASIDWLAQRNNVAPDELYTPHSWLLRVVYALLNLYEHHHSAHLRQRAVQKLYEHIVADDRFTKSISIGPISKTINMLVRWYVDGPASTAFQEHVSRIPDYLWMGLDGMKMQGTNGSQIWDTAFAIQALLEAGGHHRPEFSSCLQKAHEFLRLSQVPDNPPDYQKYYRQMRKGGFSFSTLDCGWIVSDCTAEALKAVLLLQEKCPHVTEHIPRERLCDAVAVLLNMRNPDGGFATYETKRGGHLLELLNPSEVFGDIMIDYTYVECTSAVMQALKYFHKRFPEHRAAEIRETLTQGLEFCRRQQRADGSWEGSWGVCFTYGTWFGLEAFACMGQTYRDGTACAEVSRACDFLLSRQMADGGWGEDFESCEERRYLQSAQSQIHNTCWAMMGLMAVRHPDIEAQERGVRCLLEKQLPNGDWPQENIAGVFNKSCAISYTSYRNIFPIWALGRFSQLYPERALAGHP

SEQ ID NO: 9 - DNA, 1168 bp, Homo sapiens
  CDS (30)..(800)
gagctggaagtgagagcagatccctaaccatgagcaccagccaaccaggggcctgcccatgccagggagctgcaagccgccccgccattctctacgcacttctgagctccagcctcaaggctgtcccccgaccccgtagccgctgcctatgtaggcagcaccggcccgtccagctatgtgcacctcatcgcacctgccgggaggccttggatgttctggccaagacagtggccttcctcaggaacctgccatccttctggcagctgcctccccaggaccagcggcggctgctgcagggttgctggggccccctcttcctgcttgggttggcccaagatgctgtgacctttgaggtggctgaggccccggtgcccagcatactcaagaagattctgctggaggagcccagcagcagtggaggcagtggccaactgccagacagaccccagccctccctggctgcggtgcagtggcttcaatgctgtctggagtccttctggagcctggagcttagccccaaggaatatgcctgcctgaaagggaccatcctcttcaaccccgatgtgccaggcctccaagccgcctcccacattgggcacctgcagcaggaggctcactgggtgctgtgtgaagtcctggaaccctggtgcccagcagcccaaggccgcctgacccgtgtcctcctcacggcctccaccctcaagtccattccgaccagcctgcttggggacctcttctttcgccctatcattggagatgttgacatcgctggccttcttggggacatgcttttgctcaggtgacctgttccagcccaggcagagatcaggtgggcagaggctggcagtgctgattcagcctggccatccccagaggtgacccaatgctcctggaggggcaagcctgtatagacagcacttggctccttaggaacagctcttcactcagccacaccccacattggacttccttggtttggacacagtgctccagctgcctgggaggcttttggtggtccccacagcctctgggccaagactcctgtcccttcttgggatgagaatgaaagcttaggctgcttattggaccagaagtcctatcgactttatacagaactgaattaagttattgatttttgtaataaaaggtatgaaacactaaaaaaaaa

SEQ ID NO: 10 - protein, 257 aa, Homo sapiens
MSTSQPGACPCQGAASRPAILYALLSSSLKAVPRPRSRCLCRQHRPVQLCAPHRTCREALDVLAKTVAFLRNLPSFWQLPPQDQRRLLQGCWGPLFLLGLAQDAVTFEVAEAPVPSILKKILLEEPSSSGGSGQLPDRPQPSLAAVQWLQCCLESFWSLELSPKEYACLKGTILFNPDVPGLQAASHIGHLQQEAHWVLCEVLEPWCPAAQGRLTRVLLTASTLKSIPTSLLGDLFFRPIIGDVDIAGLLGDMLLLR

SEQ ID NO: 11 - DNA, 489 bp, Homo sapiens
  CDS (43)..(423)
agagccgcaggtcagtcgtgaagagggagctctattgccaccatgagtttctccggcaagtaccaactgcagagccaggaaaactttgaagccttcatgaaggcaatcggtctgccggaagagctcatccagaaggggaaggatatcaagggggtgtcggaaatcgtgcagaatgggaagcacttcaagttcaccatcaccgctgggtccaaagtgatccaaaacgaattcacggtgggggaggaatgtgagctggagacaatgacaggggagaaagtcaagacagtggttcagttggaaggtgacaataaactggtgacaactttcaaaaacatcaagtctgtgaccgaactcaacggcgacataatcaccaataccatgacattgggtgacattgtcttcaagagaatcagcaagagaatttaaacaagtctgcatttcatattattttagtgtgtaaaattaatgtaataaagtgaactttgtttt

SEQ ID NO: 12 - protein, 127 aa, Homo sapiens
MSFSGKYQLQSQENFEAFMKAIGLPEELIQKGKDIKGVSEIVQNGKHFKFTITAGSKVIQNEFTVGEECELETMTGEKVKTVVQLEGDNKLVTTFKNIKSVTELNGDIITNTMTLGDIVFKRISKRI

SEQ ID NO: 13 - DNA, 1783 bp, Homo sapiens
  CDS (246)..(1496)
  sig_peptide (246)..(731)
  mat_peptide (732)..(1496)
tcgagcccgctttccagggaccctacctgagggcccacaggtgaggcagcctggcctagcaggccccacgccaccgcctctgcctccaggccgcccgctgctgcggggccaccatgctcctgcccaggcctggagactgacccgaccccggcactacctcgaggctccgcccccacctgctggaccccagggtcccaccctggcccaggaggtcagccagggaatcattaacaagaggcagtgacatggcgcagaaggagggtggccggactgtgccatgctgctccagacccaaggtggcagctctcactgcggggaccctgctacttctgacagccatcggggcggcatcctgggccattgtggctgttctcctcaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctctgaccggtggcttctcgctgcgcagcctccagggcccgaggtgatcccggtggtgggatccacgctgggccgaggatgggacgtttttcttcttgggcccggtccacaggtccaaggacaccctccctccagggtcctctcttccacagtggcgggcccactcagccccgagaccacccaacctcaccctcctgacccccatgtaaatattgttctgctgtctgggactcctgtctaggtgcccctgatgatgggatgctctttaaataataaagatggttttgatt

SEQ ID NO: 14 - protein, 417 aa, Homo sapiens
MAQKEGGRTVPCCSRPKVAALTAGTLLLLTAIGAASWAIVAVLLRSDQEPLYPVQVSSADARLMVFDKTEGTWRLLCSSRSNARVAGLSCEEMGFLRALTHSELDVRTAGANGTSGFFCVDEGRLPHTQRLLEVISVCDCPRGRFLAAICQDCGRRKLPVDRIVGGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQL

SEQ ID NO: 15 - DNA, 1534 bp, Homo sapiens
  CDS (26)..(1324)
  sig_peptide (26)..(100)
  mat_peptide (101)..(1324)
ggaattccctggagcagagttgagaatggagagaatgttacctctcctggctctggggctcttggcggctgggttctgccctgctgtcctctgccaccctaacagcccacttgacgaggagaatctgacccaggagaaccaagaccgagggacacacgtggacctcggattagcctccgccaacgtggacttcgctttcagcctgtacaagcagttagtcctgaaggcccttgataagaatgtcatcttctccccactgagcatctccaccgccttggccttcctgtctctgggggcccataataccaccctgacagagattctcaaggcctcgagttcacctcacggagacttactgaggcagaaattcactcagagcttccagcacctccgcgcaccctcaatcagttccagcgatgagctgcagctgagtatgggaaatgccatgtttgtcaaagagcaactcagtctgctggacaggttcacggaggatgccaagaggctgtatggctccgaggcctttgccactgactttcaggactcagctgcagctaagaagctcatcaacgactacgtgaagaatggaactagggggaaaatcacagatctgatcaaggaccccgactcgcagacaatgatggtcctggtgaattacatcttctttaaagccaaatgggagatgccctttgacccccaagatactcatcagtcaaggttctacttgagcaagaaaaagtgggtaatggtgcccatgatgagtttgcatcacctgactataccttacttccgggacgaggagctgtcctgcaccgtggtggagctgaagtacacaggcaatgccagcgcactcttcatcctccctgatcaagacaagatggaggaagtggaagccatgctgctcccagagaccctgaagcggtggagagactctctggagttcagagagataggtgagctctacctgccaaagttttccatctcgagggactataacctgaacgacatacttctccagctgggcattgaggaagccttcaccagcaaggctgacctgtcagggatcacaggggccaggaacctagcagtctcccaggtggtccataaggtcgtgtctgatgtatttgaggagggcacagaagcatctgctgccacagcagtcaaaatcaccctcctttctgcattagtggagacaaggaccattgtgcgtttcaacaggcccttcctgatgatcattgtccctacagacacccagaacatcttcttcatgagcaaagtcaccaatcccagcaagcctagagcttgcatcaagcagtggggctctcagtaaggaacttggaatgcaagctggatgcctgggtctctgggcacagctggcccctgtgcaccgtagtggccatggcatgtgtggccctgtctgcttatccttggaaggtgacagcgattccctgtgaagctctcacacgcacaggggcccatggactcttcagtctggagggtcctggcctcctgacagcaataaataatttcgttggcc

SEQ ID NO: 16 - protein, 433 aa, Homo sapiens
MERMLPLLALGLLAAGFCPAVLCHPNSPLDEENLTQENQDRGTHVDLGLASANVDFAFSLYKQLVLKALDKNVIFSPLSISTALAFLSLGAHNTTLTEILKASSSPHGDLLRQKFTQSFQHLRAPSISSSDELQLSMGNAMFVKEQLSLLDRFTEDAKRLYGSEAFATDFQDSAAAKKLINDYVKNGTRGKITDLIKDPDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKVVSDVFEEGTEASAATAVKITLLSALVETRTIVRFNRPFLMIIVPTDTQNIFFMSKVTNPSKPRACIKQWGSQ

SEQ ID NO: 17 - DNA, 2008 bp, Homo sapiens
  CDS (222)..(857)
tgggacactgctcagggaagagcctgctacggtggactgtgagactcagtgcactgtcctcctcccagcgaccccacgctggaccccctgccggaccctccacccttcggcccccaagcttcccaggggcttcctttggactggactgtccctgctcatccattctcctgccacccccagacctcctcagctccaggttgccacctcctctcgccagagtgatgaggtcccggcttctgctctccgtggcccatctgcccacaattcgggagaccacggaggagatgctgcttgggggtcctggacaggagcccccaccctctcctagcctggatgactacgtgaggtctatatctcgactggcacagcccacctctgtgctggacaaggccacggcccagggccaacccaggccaccccacaggccagcccaggcctgccggaagggccgccctgctgtgtccctgcgagacatcaccgcacgtttcagtggccagcagcccacactgcccatggctgatactgtggaccccctggactggctttttggggagtcccaggaaaagcagccaagccagagggacctgccaaggaggactggcccctctgctggcctctggggtccacatagacagatggacagcagcaagcccacgggggcccccagagggaggctctgtgaagccaggatgcctgggcattccctggcaagaccaccgcaggatgggcagcagagctctgacctaagaagctggacttttgggcagtctgcccaagccatggcctcccgccaccgcccccgccccagcagtgtcctcagaacactctactcgcacctcccggtgatccatgaactctgacccctccccagtaaaggcttctgtagagagcatgctgggtctgcatctcctctcgtctcctccatggtggtcactgcccctggcaggtctctgaaagggaaatgcttttctgcggaggcccctgcttgggcagttcacagtgagaccgaccccctctgaatatgataacagcctgtttcacatgaggagatgttaccaatcccgttcgctctgacccttgctggctgatcaccttgagcaacttacttaacatctgtgttcctcagtttctcatgggtaatatagggataattactggcacctgcctcccaggccattctgacgtgtaaccgcatataggagcccactggctgagtagctaccatcatcgctggtggggaaactggtggtaggggtgtgagggtagtgggggtgtcagccccccaggtgtttcagaacaaggcctcgggcactcccaagtctgcctcttggctcccaccctcaaagcccatgttctgcgaggcccaagagaacacatggagtcttagcaaatgcactaatgtattccgggggactgtcacctggcaccactggggcactctgctggctacaactcatacgtcctgtggtggcattgggagagttcccccatgatgagggccaagatagaatctgtaccactcagtgctaccatccccacccctacaccacttccacacaggggcctcatggcatggtcagggtcccagctgtaggtgagagcagggcactgtccagctgtccactggggaagtcaagatgtcctaaggcccaggtcagggcatctggagtctgaaggaccctagttcctagaggcatctggcagcaagaaggtgaggcatcagggaacgggaatcaggctgggactgatcagaggtgaagggacagagagaggagaggaggaagattgagctgggggcaacagccaagctcacctgggcaggtctctgccacctccttgctctgtgagctgtcagtctaggttattctctttttttgtggctatttttaattgctttggatttgttaaatgttttctgtcttctgttaagtgtgttttctctggagatagaatgtaaaccatattaaaaggaaaaagtttcagacaagcaaaaaaaaaaaaaaaaaaa

SEQ ID NO: 18 - protein, 212 aa, Homo sapiens
MRSRLLLSVAHLPTIRETTEEMLLGGPGQEPPPSPSLDDYVRSISRLAQPTSVLDKATAQGQPRPPHRPAQACRKGRPAVSLRDITARFSGQQPTLPMADTVDPLDWLFGESQEKQPSQRDLPRRTGPSAGLWGPHRQMDSSKPTGAPRGRLCEARMPGHSLARPPQDGQQSSDLRSWTFGQSAQAMASRHRPRPSSVLRTLYSHLPVIHEL

SEQ ID NO: 19 - DNA, 1649 bp, Homo sapiens
  CDS (367)..(1068)
agcgagcggggccagcgctgcagaaggcggcggctggctctccgggacggtcacatcccgctgcaggggcgggcggaggccgccgcactgcctcccgcaccggggacccaggccagcgtccgggcaacgccccctgctcccggacagactccgtggcccgctcgagccctgggggctccgcagacccgcgcccgctccgcccgcagctcggccccgcgctgcccgcgtcgccgggcccgcgccgggatggggtaggggcagcgccaccgagtcgggcgatgggccgccctctgggcaccgagcagccccccgaggcctgaccaaccgcgaggaccggcggaggagccccgcctggatgtcaagcggatgccaagcggatgccacagttccccccccagcggactccgtggggacatggcttcgctggtgcccctttccccatatctaagccccacggtcctcctgctggtcagctgtgacctgggcttcgtgcgagcagaccggcctccctctcctgtgaatgtgacggtcactcacctcagagccaactcggccactgtgtcctgggacgtcccagaaggcaacatcgtcattggctactccatttcccagcaacggcagaatggccccgggcagcgtgtgattcgggaggtgaacaccaccacccgggcctgtgccctctggggcctggctgaagacagtgactacacagtgcaggtcaggagcatcggccttcggggagagagtcccccagggccccgggtgcacttccgaactctcaagggttctgaccggctaccttcaaacagttcaagcccaggtgacatcacagtggaaggtctggatggagagcggccactgcagactggggaagtggtcatcattgtggtggtgttgctcatgtgggctgctgtaattgggctgttctgccgtcagtatgacatcatcaaggacaatgactccaacaacaatcccaaggagaagggaaaggggccggaacagagtcctcagggaaggccagtggggacaagacagaaaaagtcaccatctatcaacaccatcgacgtttgagtgaagaaacacacccagaagagagatgcactaacaactggggatagggatggggtcagggggagcccaagatggtgatctgcccgagactcccagagggtaatgccactcccacaatctcaggcctggtacccatcctctttccactgtgagcagagccagaaggtaggtctgttcagagtctgtgcccctggacctggggagtggatatcagatgggatatctccttccattccccggtccaggggagagtcactagttgtaccctactccattaggtcccaaatgggggccccatttcacctgtatcaggactctgagcatccccagctgccccacatcttgcctctggccctcagagaggggtgtttctgtgggtactcctcttaccccagcaaataaaaggaattgtctgaccctagaggcagatgctgcactgcactactccaatgtcttccatggagcctcaggtgctccccctctcacctggcagccccttcagctgctagtgatatcacttgttggacatttttccaataaaggttcttggacaaactggaaaaaaaaaaaaaaaaaaa

SEQ ID NO: 20 - protein, 234 aa, Homo sapiens
MPSGCHSSPPSGLRGDMASLVPLSPYLSPTVLLLVSCDLGFVRADRPPSPVNVTVTHLRANSATVSWDVPEGNIVIGYSISQQRQNGPGQRVIREVNTTTRACALWGLAEDSDYTVQVRSIGLRGESPPGPRVHFRTLKGSDRLPSNSSSPGDITVEGLDGERPLQTGEVVIIVVVLLMWAAVIGLFCRQYDIIKDNDSNNNPKEKGKGPEQSPQGRPVGTRQKKSPSINTIDV

SEQ ID NO: 21 - DNA, 3915 bp, Homo sapiens
  CDS (243)..(1730)
gtggggtggggtggggctgggggcttgtcgccctttcaggctccaccctttgcggagattataaatagtcatgatcccagcgagacccagagatgcctgtaatggtgagactttggatccttcctgaggacgtggagaaaactttctgctgagaaggacattttgaaggttttgttggctgaaaaagctgtttctggaatcacccctagatctttcttgaagacttgaattagattacagcgatggggacacagaaggtcaccccagctctgatatttgccatcacagttgctacaatcggctctttccaatttggctacaacactggggtcatcaatgctcctgagaagatcataaaggaatttatcaataaaactttgacggacaagggaaatgccccaccctctgaggtgctgctcacgtctctctggtccttgtctgtggccatattttccgtcgggggtatgatcggctccttttccgtcggactcttcgtcaaccgctttggcaggcgcaattcaatgctgattgtcaacctgttggctgtcactggtggctgctttatgggactgtgtaaagtagctaagtcggttgaaatgctgatcctgggtcgcttggttattggcctcttctgcggactctgcacaggttttgtgcccatgtacattggagagatctcgcctactgccctgcggggtgcctttggcactctcaaccagctgggcatcgttgttggaattctggtggcccagatctttggtctggaattcatccttgggtctgaagagctatggccgctgctactgggttttaccatccttcctgctatcctacaaagtgcagcccttccattttgccctgaaagtcccagatttttgctcattaacagaaaagaagaggagaatgctaagcagatcctccagcggttgtggggcacccaggatgtatcccaagacatccaggagatgaaagatgagagtgcaaggatgtcacaagaaaagcaagtcaccgtgctagagctctttagagtgtccagctaccgacagcccatcatcatttccattgtgctccagctctctcagcagctctctgggatcaatgctgtgttctattactcaacaggaatcttcaaggatgcaggtgttcaagagcccatctatgccaccatcggcgcgggtgtggttaatactatcttcactgtagtttctctatttctggtggaaagggcaggaagaaggactctgcatatgataggccttggagggatggctttttgttccacgctcatgactgtttctttgttattaaaggataactataatgggatgagctttgtctgtattggggctatcttggtctttgtagccttctttgaaattggaccaggccccattccctggtttattgtggccgaactcttcagccagggcccccgcccagctgcgatggcagtggccggctgctccaactggacctccaacttcctagtcggattgctcttcccctccgctgctcactatttaggagcctacgtttttattatcttcaccggcttcctcattaccttcttggcttttaccttcttcaaagtccctgagacccgtggcaggacttttgaggatatcacacgggcctttgaagggcaggcacacggtgcagatagatctggaaaggacggcgtcatggagatgaacagcatcgagcctgctaaggagaccaccaccaatgtctaagtcgtgcctccttccacctccctcccggcatgggaaagccacctctccctcaacaagggagagacctcatcaggatgaacccaggacgcttctgaatgctgctacttaattcctttctcatcccacgcactccatgagcaccccaaggctgcggtttgttggatcttcaatggctttttaaattttatttcctggacatcctcttctgcttaggagagaccgagtgaacctaccttcatttcaggagggattggccgcttggcacatgacaactttgccagcttttcctcccttgggttctgatattgccgcactaggggatataggagaggaaaagtaaggtgcagttcccccaacctcagacttaccaggaagcagatacatatgagtgtggaagccggagggtgtttatgtaagagcaccttcctcacttccatacagctctacgtggcaaattaacttgagttttatttattttatcctctggtttaattacataatttttttttttttactttaagtttcaggatacatgtgccgaatgtgcaggtttgttacataggtatatatatgccatgatggaaatatttatttttttaagcgtaattttgccaaataataaaaacagaaggaaattgagattagagggaggtgtttaaagagaggttatagagtagaagatttgatgctggagaggttaaggtgcaataagaatttagggagaaatgttgttcattattggagggtaaatgatgtggtgcctgaggtctgtacgttacctcttaacaatttctgtccttcagatggaaactctttaacttctcgtaaaagtcatatacctatataataaagctactgatttccttggagcttttttctttaagataatagtttacatgtagtagtacttgaaatctaggattattaactaatatgggcattgtagttaatgatggttgatgggttctaattttggatggagtccagggaagagaaagtgatttctagaaagcctgttcccctcactggatgaaataactccttcttgtagtagtctcattacttttgaagtaatcccgccacctatctcgtgggagagccatccaaataagaaacctaaaataattggttcttggtagagattcattatttttccactttgttctttaggagattttaggtgttgattttctgttgtattttaactcatacctttaaaggaattccccaaagaatgtttatagcaaacttggaatttgtaacctcagctctgggagaggatttttttctgagcgattattatctaaagtgtgttgttgctttaggctcacggcacgcttgcgtatgtctgttaccatgtcactgtggtcctatgccgaatgccctcaggggacttgaatctttccaataaaccaggtttagacagtatgagtcaatgtgcagtgtagcccacacttgagaggatgaatgtatgtgcactgtcactttgctctgggtggaagtacgttattgttgacttattttctctgtgtttgttcctacagcccctttttcatatgttgctcagtctccctttcccttcttggtgcttacacatctcagaccctttagccaaacccttgtcagtgacagtattttggttcttagttctcactgttccctctgctcctggagcctttgaataaaaatgcacgtagctgaggccggatgcggtggctcacgcctgtaatcccagcactttgggaggcctaggcgggcggtcaggggttcgagaccagtctggccaacatcgtgaaaccctgtctctactaaaaatgcaaaaattagccgggcgtggtggcgggcgcctgtaatcccagctacttgggaagctgaggcgggagaatcatgtgaacccgggacgcaggggttgcagtgagcggagatcgcatcattgcactctagcctgggccacagggcgagactccgtctcaaaaaaaaaaaaatgcacatagctatcgagtgtgctttagcttgaaaaggtgaccttgcaacttcatgtcaactttctggctcctcaaacagtaggttggcagtaaggcagggtcccatttctcactgagaagattgtgaatatttccatatggattttctattgttactctggttctttgttttaaaataaaaattctgaatgtacacg

SEQ ID NO: 22 - protein, 496 aa, Homo sapiens
MGTQKVTPALIFAITVATIGSFQFGYNTGVINAPEKIIKEFINKTLTDKGNAPPSEVLLTSLWSLSVAIFSVGGMIGSFSVGLFVNRFGRRNSMLIVNLLAVTGGCFMGLCKVAKSVEMLILGRLVIGLFCGLCTGFVPMYIGEISPTALRGAFGTLNQLGIVVGILVAQIFGLEFILGSEELWPLLLGFTILPAILQSAALPFCPESPRFLLINRKEEENAKQILQRLWGTQDVSQDIQEMKDESARMSQEKQVTVLELFRVSSYRQPIIISIVLQLSQQLSGINAVFYYSTGIFKDAGVQEPIYATIGAGVVNTIFTVVSLFLVERAGRRTLHMIGLGGMAFCSTLMTVSLLLKDNYNGMSFVCIGAILVFVAFFEIGPGPIPWFIVAELFSQGPRPAAMAVAGCSNWTSNFLVGLLFPSAAHYLGAYVFIIFTGFLITFLAFTFFKVPETRGRTFEDITRAFEGQAHGADRSGKDGVMEMNSIEPAKETTTNV

SEQ ID NO: 23 - DNA, 1085 bp, Homo sapiens
  CDS (76)..(678)
atcctgtctgtccgaacccagacacaagtcttcactccttcctgcgagccctgaggaagccttctttccccagacatggccaacaagggtccttcctatggcatgagccgcgaagtgcagtccaaaatcgagaagaagtatgacgaggagctggaggagcggctggtggagtggatcatagtgcagtgtggccctgatgtgggccgcccagaccgtgggcccttgggcttccaggtgtggctgaagaatggcgtgattctgagcaagctggtgaacagcctgtaccctgatggctccaagccggtgaaggtgcccgagaacccaccctccatggtcttcaagcagatggagcaggtggctcagttcctgaaggcggctgaggactctggggtcatcaagactgacatgttccagactgttgacctctttgaaggcaaagacatggcagcagtgcagaggaccctgatggctttgggcagcttggcagtgaccaagaatgatgggcactaccgtggagatcccaactggtttatgaagaaagcgcaggagcataagagggaattcacagagagccagctgcaggagggaaagcatgtcattggccttcagatgggcagcaacagaggggcctcccaggccggcatgacaggctacggacgacctcggcagatcatcagttagagcggagagggctagccctgagcccggcgctcccccagctccttggctgcagccatcccgcttagcctgcctcacccacacccgtgtggtaccttcagccctggccaagctttgaggctctgtcactgagcaatggtaactgcacctgggcagctcctccctgtgcccccagcctcagcccaacttcttacccgaaagcatcactgccttggcccctccctcccggcggcccccatcacctctactgtctcctccctgggctaagcaggggagaagcgggctgggggtagcctggatgtgggcgaagtccactgtcctccttggcggcaaaagcccattgaagaagaaccagcccagcctgccccctatcttgtacctggaatatttttggggttggaactctc

SEQ ID NO: 24 - protein, 201 aa, Homo sapiens
MANKGPSYGMSREVQSKIEKKYDEELEERLVEWIIVQCGPDVGRPDRGPLGFQVWLKNGVILSKLVNSLYPDGSKPVKVPENPPSMVFKQMEQVAQFLKAAEDSGVIKTDMFQTVDLFEGKDMAAVQRTLMALGSLAVTKNDGHYRGDPNWFMKKAQEHKREFTESQLQEGKHVIGLQMGSNRGASQAGMTGYGRPRQIIS

SEQ ID NO: 25 - DNA, 30 bp, Artificial
  Oligonucleotide designed to act as primer for amplifying kiaa1001 gene transcript.
ggaacatctctttgaattgtatttcttgta

SEQ ID NO: 26 - DNA, 22 bp, Artificial
  Oligonucleotide designed to act as primer for amplifying kiaa1001 gene transcript.
agccacagccaaaaaagacttt

SEQ ID NO: 27 - DNA, 32 bp, Artificial
  Oligonucleotide designed to act as TaqMan probe for detecting amplification of kiaa1001 gene transcript.
ttacatacttagagagagactcttttagccag

SEQ ID NO: 28 - DNA, 28 bp, Artificial
  Oligonucleotide designed to act as primer for amplifying asah1 gene transcript.
accctaaggaagttgctaacttaaaaaa

SEQ ID NO: 29 - DNA, 29 bp, Artificial
  Oligonucleotide designed to act as primer for amplifying asah1 gene transcript.
tccacaagtctttgacttgtttatttact

SEQ ID NO: 30 - DNA, 23 bp, Artificial
  Oligonucleotide designed to act as TaqMan probe for detecting amplification of asah1 gene transcript.
ctgcatcccacgttctgttaatt

SEQ ID NO: 31 - DNA, 22 bp, Artificial
  Oligonucleotide designed to act as primer for amplifying mgc4171 gene transcript.
caggtggtcttttggtgcctta

SEQ ID NO: 32 - DNA, 18 bp, Artificial
  Oligonucleotide designed to act as primer for amplifying mgc4171 gene transcript.
agtggctcccacgctgaa

SEQ ID NO: 33 - DNA, 24 bp, Artificial
  Oligonucleotide designed to act as TaqMan probe for detecting amplification of mgc4171 gene transcript.
tgaagagtcggattttgaagcagc

SEQ ID NO: 34 - DNA, 24 bp, Artificial
  Oligonucleotide designed to act as primer for amplifying lss gene transcript.
gtccggtgtctacttgagaaacag

SEQ ID NO: 35 - DNA, 21 bp, Artificial
  Oligonucleotide designed to act as primer for amplifying lss gene transcript.
agaccccagcaatgttttcct

SEQ ID NO: 36 - DNA, 18 bp, Artificial
  Oligonucleotide designed to act as TaqMan probe for detecting amplification of lss gene transcript.
cccaatggcgactggccg

SEQ ID NO: 37 - DNA, 21 bp, Artificial
  Oligonucleotide designed to act as primer for amplifying nr0b2 gene transcript.
cagcacttggctccttaggaa

SEQ ID NO: 38 - DNA, 23 bp, Artificial
  Oligonucleotide designed to act as primer for amplifying nr0b2 gene transcript.
actgtgtccaaaccaaggaagtc

SEQ ID NO: 39 - DNA, 23 bp, Artificial
  Oligonucleotide designed to act as TaqMan probe for detecting amplification of nr0b2 gene transcript.
agctcttcactcagccacacccc

SEQ ID NO: 40 - DNA, 22 bp, Artificial
  Oligonucleotide designed to act as primer for amplifying fabp1 gene transcript.
gagtttctccggcaagtaccaa

SEQ ID NO: 41 - DNA, 20 bp, Artificial
  Oligonucleotide designed to act as primer for amplifying fabp1 gene transcript.
cagaccgattgccttcatga

SEQ ID NO: 42 - DNA, 25 bp, Artificial
  Oligonucleotide designed to act as TaqMan probe for detecting amplification of fabp1 gene transcript.
tgcagagccaggaaaactttgaagc

SEQ ID NO: 43 - DNA, 22 bp, Artificial
  Oligonucleotide designed to act as primer for amplifying hpn gene transcript.
gaaaccagatcaagcccaagat

SEQ ID NO: 44 - DNA, 18 bp, Artificial
  Oligonucleotide designed to act as primer for amplifying hpn gene transcript.
ccctggcaggcatcaatg

SEQ ID NO: 45 - DNA, 20 bp, Artificial
  Oligonucleotide designed to act as TaqMan probe for detecting amplification of hpn gene transcript.
ttctgtgctggctaccccga

SEQ ID NO: 46 - DNA, 21 bp, Artificial
  Oligonucleotide designed to act as primer for amplifying serpina3 gene transcript.
gaggagggcacagaagcatct

SEQ ID NO: 47 - DNA, 24 bp, Artificial
  Oligonucleotide designed to act as primer for amplifying serpina3 gene transcript.
ccttgtctccactaatgcagaaag

SEQ ID NO: 48 - DNA, 24 bp, Artificial
  Oligonucleotide designed to act as TaqMan probe for detecting amplification of serpina3 gene transcript.
tgccacagcagtcaaaatcaccct

SEQ ID NO: 49 - DNA, 20 bp, Artificial
  Oligonucleotide designed to act as primer for amplifying depp gene transcript.
tgtggtggcattgggagagt

SEQ ID NO: 50 - DNA, 25 bp, Artificial
  Oligonucleotide designed to act as primer for amplifying depp gene transcript.
tggtagcactgagtggtacagattc

SEQ ID NO: 51 - DNA, 22 bp, Artificial
  Oligonucleotide designed to act as TaqMan probe for detecting amplification of depp gene transcript.
cccccatgatgagggccaagat

SEQ ID NO: 52 - DNA, 22 bp, Artificial
  Oligonucleotide designed to act as primer for amplifying flj22362 gene transcript.
ggtaatgccactcccacaatct

SEQ ID NO: 53 - DNA, 21 bp, Artificial
  Oligonucleotide designed to act as primer for amplifying flj22362 gene transcript.
ccttctggctctgctcacagt

SEQ ID NO: 54 - DNA, 23 bp, Artificial
  Oligonucleotide designed to act as TaqMan probe for detecting amplification of flj22362 gene transcript.
aggcctggtacccatcctctttc

SEQ ID NO: 55 - DNA, 21 bp, Artificial
  Oligonucleotide designed to act as primer for amplifying slc2a3 gene transcript.
gcttgaaaaggtgaccttgca

SEQ ID NO: 56 - DNA, 23 bp, Artificial
  Oligonucleotide designed to act as primer for amplifying slc2a3 gene transcript.
tgccttactgccaacctactgtt

SEQ ID NO: 57 - DNA, 23 bp, Artificial
  Oligonucleotide designed to act as TaqMan probe for detecting amplification of slc2a3 gene transcript.
tcatgtcaactttctggctcctc

SEQ ID NO: 58 - DNA, 25 bp, Artificial
  Oligonucleotide designed to act as primer for amplifying tagln gene transcript.
gagcataagagggaattcacagaga

SEQ ID NO: 59 - DNA, 20 bp, Artificial
  Oligonucleotide designed to act as primer for amplifying tagln gene transcript.
ctgttgctgcccatctgaag

SEQ ID NO: 60 - DNA, 27 bp, Artificial
  Oligonucleotide designed to act as TaqMan probe for detecting amplification of tagln gene transcript.
agctgcaggagggaaagcatgtcattg

(19) United States
(12) Patent Application Publication
(10) Pub. No.: US 2007/0218460 A1
(43) Pub. Date: Sep. 20, 2007

(54) Method of Estimating Toxicity of Drug

(75) Inventors: Kenji Takami, Osaka-shi (JP); Hiroshi Sawada, Osaka-shi (JP); Satoru Asahi, Osaka-shi (JP)

Correspondence Address: EDWARDS ANGELL PALMER & DODGE LLP, P.O. BOX 55874, BOSTON, MA, 02205, US

(73) Assignee: TAKEDA PHARMACEUTICAL COMPANY LIMITED, OSAKA (JP)

(21) Appl. No.: 10/581,008

(22) PCT Filed: Nov. 26, 2004
(86) PCT No.: PCT/JP04/17995

§ 371 Date: Jan. 9, 2007

(30) Foreign Application Priority Data
Nov. 27, 2003 (JP) 2003-397551

(51) Int. Cl. C12Q 1/68

(52) U.S. Cl. 435/6

(57) ABSTRACT

The present invention provides a novel in vitro prediction system of a phospholipidosis inducing potential of a novel drug-caused phospholipidosis marker gene and a drug. Specifically, the invention provides a reagent for prediction of a phospholipidosis inducing potential of a compound containing a nucleic acid capable of detecting the expression of a gene having the same or substantially the same base sequence as the base sequence shown in SEQ ID NO:1, 3, 5, 7, 9, 11, 13, 15, 17, 19, 21 or 23. Furthermore, the invention provides a method for predicting a phospholipidosis inducing potential of a compound, which includes detecting the expression of two or more genes showing expression variation in correlation with phospholipidosis expression in a mammalian cell exposed to a test compound.

### TECHNICAL FIELD

The present invention relates to a method for predicting the toxicity of drugs and tools therefor. More particularly, the present invention relates to a method for predicting the toxicity of pharmaceutical candidate compounds (e.g., phospholipidosis induction potential etc.) with expression variations of marker genes as indices, and reagents, kits and the like for the detection of the toxicity marker genes.

### BACKGROUND ART

In recent years, the bottleneck up to the optimization of a lead compound in drug discovery research has been shifting from the efficacy screening to the toxicity screening as a result of the introduction of combinatorial chemistry and high-throughput screening. Therefore, establishment of highly efficient toxicity evaluation or toxicity prediction system capable of contributing to the obliteration of such bottleneck is strongly demanded.

At present, toxicity evaluation of pharmaceutical candidate compounds is generally conducted by in vivo toxicity tests on administration of compounds to laboratory animals such as rat and the like. Such tests are defective in that they require (1) several days or months for the expression of toxicity, (2) compounds in large amounts and the like. Particularly, for rapid prediction of the presence or absence of toxicity and efficient optimization of structure in the initial stage of development when the synthesis amount of compound is limited, construction of an in vitro screening system capable of evaluating many samples in smaller amounts in a short time is essential.

Drug induced phospholipidosis (Phospholipidosis; hereinafter sometimes to be abbreviated as “PLsis”) is defined to be a phenomenon where phospholipid accumulates in excess in the cell, and induced by many pharmaceutical agents such as antidepressants, antianginal drugs, antimalarial drugs, anti-anorectic drugs, hypolipidemic drugs and the like or metabolites thereof. In PLsis, phospholipid is mainly accumulated in lysosome and a cyclic or elliptic myelin-like structure (lamellar body) is observed electron microscopically. While the expression mechanism of toxicity has not been completely elucidated, it is considered to be caused by 1) inhibition of lysosomal enzyme (mainly phospholipid degradation enzyme (phospholipase)) activity by compound, 2) inhibition of transport pathway involved in phospholipid metabolism by compound, 3) inhibition of degradation of complex by the formation of a complex of compound and phospholipid, 4) promotion of synthesis of phospholipid by compound and the like.

Many of the PLsis-inducing compounds have a structure comprising, in a molecule, a hydrophobic domain and a positively electrically charged hydrophilic domain in combination (cationic amphiphilic drug; CAD). In recent years, along with the progress of genome analysis, the value of orphan receptors as drug discovery targets has been recognized and the development of agonists or antagonists against the receptors is being undertaken. However, because such compounds act on receptors, they mostly have CAD structures and an increasing number of incidents have been experienced where expression of PLsis prevents development of pharmaceutical products. Therefore, the development of an efficient, evaluation or prediction system of PLsis inducibility has been urgently desired.

In response, an evaluation method using phospholipase activity inhibition as an index (Matsuzawa, Y. and Hostetler, K. Y., J. Biol. Chem. (US), 1980, vol. 255 (No. 2), pp. 646-652), a detection method of phospholipid accumulation in hepatocyte or cultured lymphocyte using a fluorescence dye (Gum, R. J. et al., Biochem. Pharmacol. (UK), 2001, Vol. 62, pp. 1661-1673 and Xia, Z. et al., Biochem. Pharmacol. (UK), 1997, Vol. 53, pp. 1521-1532) and the like have been proposed. However, all of them are insufficient in terms of reliability and/or rapid performance and the like, and a practical in vitro screening system has not been established yet.

Incidentally, microarray technology for simultaneously monitoring expression of several thousand to several tens of thousand kinds of mRNAs (comprehensive gene expression analysis, transcriptomics) has been actively used in various kinds of fields of medicine and biology. In the field toxicology, too, this technology has been utilized for the elucidation of the mechanism of toxicity expression and the study of toxicity prediction, and is expected to be a new study field called toxicogenomics (Aardema, M. J. and MacGregor, J. T. et al., Mutat. Res. (Netherlands), 2002, Vol. 499, pp. 13-25). Toxicity phenomenon is considered to accompany not only independent changes in one or several genes, but also integral variations where many genes are correlated, such as interaction of genes, cascade and the like. Based thereon, it is expected that the behavior of molecules involved in toxicity expression can be comprehensively perceived by the use of a technique of microarray capable of analysis at transcriptome level. For example, WO02/10453 and WO02/095000 disclose methods to predict hepatotoxicity or kidney toxicity of a test compound, which comprises examining an expression amount of not less than 2 to 100 genes selected from an enormous group of genes in the presence of a test compound and comparing the results thereof with positive average and/or negative average expression amount(s) of respective genes previously calculated using known positive and negative compounds.

### DISCLOSURE OF THE INVENTION

It is an object of the present invention to provide a high-throughput method for predicting a PLsis induction potential, which comprises identifying a gene showing varying expression in correlation with PLsis expression, or a PLsis marker gene, and using expression variation of the gene as an index.

Another object of the present invention is to provide an optimization method for constructing an evaluation system capable of comprehensively perceiving a series of gene groups showing common varying expressions due to the expression of certain toxicity, and more precisely predicting the presence or absence of toxicity of a drug from the information obtained by a comprehensive analysis of expression of these genes.

As a result of a comprehensive analysis using a microarray of gene expression in human cultured cells exposed to various known PLsis-inducing compounds, the present inventors identified genes that showed remarkably varying expression for most of these compounds. From these, they extracted 12 genes having different functions and high expression variation rates and fully examined them by real-time quantitative PCR. As a result, these genes were confirmed to show varying expression in correlation with the level of emergence of a myelin-like structure or a structure having a high electron density, which is an early stage image thereof, by electromicroscopic observation. Furthermore, the present inventors perceived PLsis expression in correlation with comprehensive behavior of these marker genes, introduced the conception of average variation rate of expression, and succeeded in constructing a highly reliable in vitro evaluation system of a PLsis induction potential, which is associated with an extremely low false-positive and false-negative probability, which resulted in the completion of the present invention.

Accordingly, the present invention provides
[1] a reagent for predicting a phospholipidosis induction potential of a compound, which comprises a nucleic acid capable of hybridizing to a nucleic acid having a base sequence shown by any of SEQ ID NOs:1, 3, 5, 7, 9, 11, 13, 15, 17, 19, 21 and 23 under high stringent conditions and/or a nucleic acid capable of hybridizing to a nucleic acid having a base sequence complementary to the base sequence under high stringent conditions;
[2] a kit for predicting a phospholipidosis induction potential of a compound, which comprises one or more reagents containing a nucleic acid capable of hybridizing to a transcription product of a gene showing varying expression in correlation with expression of phospholipidosis under high stringent conditions and/or a nucleic acid capable of hybridizing to a nucleic acid having a base sequence complementary to the transcription product under high stringent conditions, wherein, when two or more reagents are contained, each reagent can detect expression of different genes;
[3] the kit of the above-mentioned [2], wherein at least one reagent comprises a nucleic acid capable of hybridizing to a nucleic acid having a base sequence shown by any of SEQ ID NOs:1, 3, 5, 7, 9, 11, 13, 15, 17, 19, 21 and 23 under high stringent conditions and/or a nucleic acid capable of hybridizing to a nucleic acid having a base sequence complementary to the base sequence under high stringent conditions;
[4] the kit of the above-mentioned [2], wherein a prediction hitting ratio of the phospholipidosis induction potential is not less than about 70% when a mammalian cell is exposed to a test compound, using an average variation rate of expression of a nucleic acid, to which the nucleic acid contained in each reagent is capable of hybridizing, in said cell as an index;
[5] a method for predicting a phospholipidosis induction potential of a compound, which comprises detecting expression variation of one or more genes showing expression variation in correlation with phospholipidosis expression, in a sample containing a mammalian cell exposed to the compound or a sample taken from a mammal administered with the compound;
[6] the method of the above-mentioned [5], wherein at least one gene has the same or substantially the same base sequence as the base sequence shown by any of SEQ ID NOs:1, 3, 5, 7, 9, 11, 13, 15, 17, 19, 21 and 23;
[7] a method for determining the standard for the judgment of the presence or absence of a phospholipidosis induction potential of a compound, which comprises
(1) detecting expression variation of one or more genes showing expression variation in correlation with phospholipidosis expression, in samples containing a mammalian cell exposed to each of two or more known phospholipidosis-inducing compounds and two or more known phospholipidosis non-inducing compounds or samples taken from mammals administered with each of said compounds, and
(2) using, as a standard value, an average variation rate capable of correctly judging the presence or absence of a phospholipidosis induction potential of the above-mentioned compounds by not less than about 70% based on the relationship between an average expression variation rate of the genes and the phospholipidosis induction potential;
[8] the method of the above-mentioned [7], wherein at least one gene has the same or substantially the same base sequence as the base sequence shown by any of SEQ ID NOs:1, 3, 5, 7, 9, 11, 13, 15, 17, 19, 21 and 23;
[9] the method of the above-mentioned [7], further comprising examining validity of the standard value using other known phospholipidosis inducing compound and known phospholipidosis non-inducing compound;
[10] the method of the above-mentioned [5], comprising comparing the average variation rate of gene expression with the standard value obtained by the method of the above-mentioned [7] or [9];
[11] a method for predicting the toxicity of a compound, which comprises,
(1) detecting expression variation of one or more genes showing expression variation in correlation with toxicity expression, in a sample containing a mammalian cell exposed to the compound or a sample taken from a mammal administered with the compound, and
(2) judging the presence or absence of toxicity of the compound with an average variation rate of the gene expression as an index;
   and the like.

### BRIEF DESCRIPTION OF THE DRAWINGS

FIG. 1 shows structural formulas, molecular weights, efficacy and addition concentrations of PLsis-inducing compounds (amiodarone, amitriptyline, AY-9944, chlorcyclizine, chlorpromazine and clomipramine).

FIG. 2 shows structural formulas, molecular weights, efficacy and addition concentrations of PLsis-inducing compounds (fluoxetine, imipramine, perhexiline, tamoxifen, thioridazine and zimelidine).

FIG. 3 shows structural formulas, molecular weights, efficacy and addition concentrations of PLsis-inducing compounds (clozapine, ketoconazole, loratadine, pentamidine and sertraline).

FIG. 4 shows structural formulas, molecular weights, efficacy and addition concentrations of PLsis non-inducing compounds (acetaminophen, clarithromycin, disopyramide, erythromycin, flecainide and haloperidol).

FIG. 5 shows structural formulas, molecular weights, efficacy and addition concentrations of PLsis non-inducing compounds (levofloxacin, ofloxacin, procainamide, quinidine, sotalol, sulfamethoxazole and sumatryptan).

FIG. 6 shows a correlation between an average variation rate of PLsis marker gene expression in HepG2 cells at 24 hr after addition of compound (vertical axis) and the frequency of emergence of a myelin-like structure in HepG2 cells at 72 hr after addition of compound (axis of abscissas), wherein +++: large myelin-like structure is found in plurality; ++: medium myelin-like structure is found in a small number; +: minor myelin-like structure is found in a small number; −: myelin-like structure is not found.

FIG. 7 shows reproducibility of the average variation rates of PLsis marker gene expression in HepG2 cells at 24 hr after addition of various compounds, wherein the axis of abscissas shows an average variation rate obtained by the first experiment, and the vertical axis shows an average variation rate obtained by the second experiment.

### BEST MODE FOR EMBODYING THE INVENTION

The present invention provides a reagent for predicting a PLsis induction potential containing a nucleic acid capable of detecting the expression of a gene showing varying expression in correlation with PLsis expression (i.e., PLsis marker gene). As used herein, the “PLsis induction potential” means an ability to produce a myelin-like structure or a structure having a high electron density, which is an early image thereof, in a target mammalian cell upon contact with the compound. Therefore, even in the case of a compound inducing PLsis by in vivo administration, when in vivo metabolite alone induces PLsis, it is PLsis induction potential negative. Even in the case of a compound to be rapidly metabolized and neutralized in the body, when the compound itself induces PLsis, it is PLsis induction potential positive.

By “varying expression in correlation with PLsis expression” is meant a statistically significant tendency toward, upon exposure of mammalian cells to various compounds, substantial increase or decrease of expression when the compound produces a myelin-like structure or a structure having a high electron density, which is an early image thereof, in the cells, and substantially no variation of expression when the compound does not produce a myelin-like structure or a structure having a high electron density, which is an early image thereof, in the cells. By the “substantial increase or decrease” is meant an increase to not less than 1.5-fold of that by non-exposure or a decrease to not more than ⅔ of that by non-exposure, and by the “substantially no variation” is meant an expression level of ⅔ to 1.5-fold of that by non-exposure.

Specifically, as the PLsis marker gene, a gene encoding a lysosomal enzyme, a gene encoding a lipid metabolism (e.g., cholesterol synthesis, fatty acid elongation, unsaturated fatty acid synthesis etc.)-related protein, a gene encoding a transport (e.g., fatty acid transport, protein transport, amino acid transport etc.)-related protein, a gene encoding a cell growth-related protein, a gene encoding a protease or protease inhibitor, a gene encoding an amino acid metabolism-related protein, and the like can be mentioned. More specifically, as a gene showing an increased expression in correlation with PLsis extracted by the present invention, human genes containing base sequences registered with IDs of NM_{—}014960, NM_{—}000859, AL518627, NM_{—}002130, AA639705, BC005807, AF116616, NM_{—}025225, U47674, D80010, NM_{—}001731, AW134535, NM_{—}004354, AF135266, AC007182, NM_{—}003832, NM_{—}019058, AB040875, AA488687, NM_{—}018687, NM_{—}021158, BG231932, NM_{—}024307, NM_{—}000235, AA873600, D63807, AF096304, AW150953, NM_{—}001360, NM_{—}021969, AC001305, NM_{—}024090, NM_{—}001443, NM_{—}006214, NM_{—}024108, NM_{—}021980, NM_{—}002151, AF003934, NM_{—}000596, U15979, M92934, NM_{—}002087, AK023348, NM_{—}002773, NM_{—}000131, BC003169, NM_{—}002217, NM_{—}003122, NM_{—}001673, NM_{—}000050, NM_{—}001085, U08024, NM_{—}003167, BC005161, AF162690, AW517464, AF116616, NM_{—}017983, AL136653, NM_{—}016061, BE966922, BE552428, NM_{—}022823, NM_{—}012445, NM_{—}000792, NM_{—}015930, NM_{—}021800, NM_{—}005980, NM_{—}000565 and AB033025 in GenBank database, and homologues thereof in other mammals and the like can be mentioned. On the other hand, as a gene showing a decreased expression in correlation with PLsis, human genes containing base sequences registered with IDs of NM_{—}006931, AL110298, NM_{—}006931, NM_{—}001955, NM_{—}003897, NM_{—}003186, AA778684, NM_{—}001283, NM_{—}012242, AI934469, NM_{—}003186 and NM_{—}002450 in GenBank database, and homologues thereof in other mammals and the like can be mentioned.

Preferably, as a PLsis marker gene in the present invention; 12 kinds of genes having the same base sequences as shown in SEQ ID NOs:1, 3, 5, 7, 9, 11, 13, 15, 17, 19, 21 and 23, or substantially the same base sequences, can be mentioned. As used herein, by the “substantially the same base sequences” is meant base sequences capable of hybridizing to nucleic acids having complementary strand sequences of base sequences shown in SEQ ID NOs:1, 3, 5, 7, 9, 11, 13, 15, 17, 19, 21 and 23 under high stringent conditions, wherein the proteins encoded thereby are the same or substantially the same proteins encoded by the base sequences shown in said SEQ ID NOs. The “high stringent conditions” refers to the conditions under which nucleic acids having complementary strand sequences of base sequences shown in SEQ ID NOs:1, 3, 5, 7, 9, 11, 13, 15, 17, 19, 21 and 23 can hybridize with nucleic acids having base sequences showing complementarity of not less than about 70%, preferably not less than about 80%, more preferably not less than about 90%, particularly preferably not less than about 95%, in the overlapping regions, and, for example, a sodium concentration of about 19-40 mM, preferably about 19-20 mM, a temperature of about 50-70° C., preferably about 60-65° C., and particularly preferably, a sodium concentration of about 19 mM and a temperature of about 65° C. can be mentioned. Those skilled in this field can easily obtain desired stringency by suitably changing a salt concentration of the hybridization solution, a temperature of hybridization reaction, a probe concentration, a probe length, a mismatch number, a hybridization reaction time, a salt concentration of the washing solution, a washing temperature, and the like.

The “substantially the same protein” refers to a protein having an amino acid sequence showing not less than about 70%, preferably not less than about 80%, more preferably not less than about 90%, particularly preferably not less than about 95%, most preferably not less than about 98%, homology to the amino acid sequence shown in SEQ ID NO:2, 4, 6, 8, 10, 12, 14, 16, 18, 20, 22 or 24 and the same level of activity as the protein having the amino acid sequence shown in the above-mentioned SEQ ID NO:2. The “same level of activity” means that the activity is qualitatively the same (e.g., physiologically or pharmacologically), and preferably, quantitatively equivalent (e.g., 0.5- to 2-fold), but may be different. As long as the homology conditions of amino acid sequence are satisfied, moreover, other quantitative elements such as molecular weight and the like may be different.

With regard to the amino acid sequence, a “homology” means a proportion (%) of the same amino acid residue and analogous amino acid residue to the whole amino acid residues overlapped in the optimal alignment (preferably, the algorithm is such that a gap can be introduced into one or both of the sequences for an optimal alignment) where two amino acid sequences are aligned using a mathematic algorithm known in the technical field. The “analogous amino acid” means amino acids having similar physiochemical properties, and for example, the amino acids are classified into groups such as an aromatic amino acid (Phe, Trp, Tyr), an aliphatic amino acid (Ala, Leu, Ile, Val), a polar amino acid (Gln, Asn), a basic amino acid (Lys, Arg, His), an acidic amino acid (Glu, Asp), an amino acid having a hydroxy group (Ser, Thr) and an amino acid having a small side-chain (Gly, Ala, Ser, Thr, Met). Substitution by such analogous amino acids is expected not to change the phenotype of proteins (i.e., conservative amino acid substitution). Specific examples of the conservative amino acid substitution are known in this technical field and described in various literatures (e.g., see Bowie et al., Science, 247: 1306-1310 (1990)).

Algorithms to determine a homology of amino acid sequence include, for example, but are not limited to, the algorithm as described in Karlin et al., Proc. Natl. Acad. Sci. USA, 90: 5873-5877 (1993) [the algorithm is incorporated into NBLAST and XBLAST programs (version 2.0) (Altschul et al., Nucleic Acids Res., 25: 3389-3402 (1997))], the algorithm as described in Needleman et al., J. Mol. Biol., 48: 444-453 (1970) [the algorithm is incorporated into a GAP program in a GCG software package], the algorithm as described in Myers and Miller, CABIOS, 4: 11-17 (1988) [the algorithm is incorporated into an ALIGN program (version 2.0) which is a part of a CGC sequence alignment software package], the algorithm as described in Pearson et al., Proc. Natl. Acad. Sci. USA, 85: 2444-2448 (1988) [the algorithm is incorporated into a FASTA program in a GCG software package], and the like.

More preferably, an amino acid sequence substantially the same as the amino acid sequence shown in SEQ ID NO:2, 4, 6, 8, 10, 12, 14, 16, 18, 20, 22 or 24 is an amino acid sequence having not less than about 70%, preferably not less than about 80%, more preferably not less than about 90%, homology to the amino acid sequence shown in any of said SEQ ID NOs.

The protein having such homology includes, for example, 1) an amino acid sequence shown in SEQ ID NO:2, 4, 6, 8, 10, 12, 14, 16, 18, 20, 22 or 24 wherein one or more (preferably about 1-30, more preferably about 1-10, particularly preferably several (1-5)) amino acids have been deleted, 2) an amino acid sequence shown in SEQ ID NO:2, 4, 6, 8, 10, 12, 14, 16, 18, 20, 22 or 24 wherein one or more (preferably about 1-30, more preferably about 1-10, particularly preferably several (1-5)) amino acids have been added, 3) an amino acid sequence shown in SEQ ID NO:2, 4, 6, 8, 10, 12, 14, 16, 18, 20, 22 or 24 wherein one or more (preferably about 1-30, more preferably about 1-10, particularly preferably several (1-5)) amino acids have been inserted, 4) an amino acid sequence shown in SEQ ID NO:2, 4, 6, 8, 10, 12, 14, 16, 18, 20, 22 or 24 wherein one or more (preferably about 1-30, more preferably about 1-10, particularly preferably several (1-5)) amino acids have been substituted by other amino acids, 5) a protein containing an amino acid sequence wherein the above-mentioned sequences have been combined, and the like.

When the amino acid sequence is inserted, deleted or substituted as mentioned above, the position of the insertion, deletion or substitution is not particularly limited.

More specifically, as a gene having a substantially the same base sequence as the base sequence shown in SEQ ID NOs:1, 3, 5, 7, 9, 11, 13, 15, 17, 19, 21 or 23, allele variants of a gene having the base sequence shown in any of these SEQ ID NOs, orthologs of the gene in non-human mammals (e.g., monkey, bovine, horse, swine, sheep, goat, dog, cat, rabbit, hamster, guinea pig, mouse, rat etc.) and the like can be mentioned.

The base sequences of human-derived PLsis marker gene of the present invention (i.e., base sequences shown in SEQ ID NOs:1, 3, 5, 7, 9, 11, 13, 15, 17, 19, 21 and 23) are all known, and published in the GenBank database under the accession Nos. of NM_{—}014960, U47674, NM_{—}024307, D63807, NM_{—}021969, NM_{—}001443, NM_{—}002151, NM_{—}001085, AL136653, NM_{—}022823, NM_{—}006931 and NM_{—}003186, respectively.

A gene having the same or substantially the same base sequence as the base sequence shown in SEQ ID NO:1 (hereinafter sometimes to be abbreviated as “kiaa1001”) encodes KIAA1001 protein, which is a lysosomal enzyme belonging to the sulfatase family.

A gene having the same or substantially the same base sequence. as the base sequence shown in SEQ ID NO:3 (hereinafter sometimes to be abbreviated as “asah1”) encodes N-acylsphingosine amidehydrolase (acidic ceramidase) 1, which is a lysosomal enzyme involved in ceramide metabolism and the lack of which causes Farber's disease (ceramide accumulation) in human.

A gene having the same or substantially the same base sequence as the base sequence shown in SEQ ID NO:5 (hereinafter sometimes to be abbreviated as “mgc4171”) encodes MGC4171 protein belonging to the glycerophosphoryl diester phosphodiesterase family involved in phospholipid degradation.

A gene having the same or substantially the same base sequence as the base sequence shown in SEQ ID NO:7 (hereinafter sometimes to be abbreviated as “lss”) encodes lanosterol synthase involved in cholesterol synthesis.

A gene having the same or substantially the same base sequence as the base sequence shown in SEQ ID NO:9 (hereinafter sometimes to be abbreviated as “nr0b2”) encodes an intranuclear receptor protein (subfamily 0, group b, member 2) involved in the regulation of expression of cholesterol 7a-hydroxylase (CYP7A1).

A gene having the same or substantially the same base sequence as the base sequence shown in SEQ ID NO:11 (hereinafter sometimes to be abbreviated as “fabp1”) encodes liver fatty acid binding protein-1 involved in lipid transport.

While the above-mentioned six genes encode lysosomal enzyme and lipid metabolism-related protein generally suggested to be related to PLsis induction, there is no report on the actual correlation between the expression of these respective genes and PLsis induction potential of a drug.

A gene having the same or substantially the same base sequence as the base sequence shown in SEQ ID NO:13 (hereinafter sometimes to be abbreviated as “hpn”) encodes hepsin, which is a transmembrane serineprotease.

A gene having the same or substantially the same base sequence. as the base sequence shown in SEQ ID NO:15 (hereinafter sometimes to be abbreviated as “serpina3”) encodes serine (cysteine) protease inhibitor (grade A, member 3).

A gene having the same or substantially the same base sequence as the base sequence shown in SEQ ID NO:17 (hereinafter sometimes to be abbreviated as “depp”) encodes a protein derived from a decidual membrane induced by progesterone.

A gene having the same or substantially the same base sequence as the base sequence shown in SEQ ID NO:19 (hereinafter sometimes to be abbreviated as “flj22362”) encodes a protein (FLJ22362) having high homology to fibronectin type III.

A gene having the same or substantially the same base sequence as the base sequence shown in SEQ ID NO:21 (hereinafter sometimes to be abbreviated as “slc2a3”) encodes a protein (family 2, member 3) belonging to a solute carrier family, which is a glucose transport carrier.

A gene having the same or substantially the same base sequence as the base sequence shown in SEQ ID NO:23 (hereinafter sometimes to be abbreviated as “tagln”) encodes transgelin, which is a cytoskeletal protein.

The proteins encoded by these six genes are generally not known to have any correlation with PLsis induction at all.

The genes having the same or substantially the same base sequence as the base sequences shown in SEQ ID NOs:1, 3, 5, 7, 9, 11, 13, 15, 17 and 19 show increased expression in correlation with PLsis expression, and the genes having the same or substantially the same base sequence as the base sequences shown in SEQ ID NO:21 and 23 show decreased expression in correlation with PLsis expression.

As a nucleic acid capable of detecting the expression of a PLsis marker gene contained in the reagent for predicting a PLsis induction potential of the present invention (hereinafter sometimes to be abbreviated as “the reagent of the present invention”), for example, a nucleic acid (probe) capable of hybridizing to a transcription product of a PLsis marker gene, an oligonucleotide set capable of functioning as a primer amplifying a part or whole of the transcription product and the like can be mentioned. That is, as the nucleic acid, for example, a nucleic acid capable of hybridizing to a nucleic acid having a base sequence shown by any of SEQ ID NOs:1, 3, 5, 7, 9, 11, 13, 15, 17, 19, 21 and 23 (sense strand=coding strand) under high stringent conditions, and/or a nucleic acid capable of hybridizing to a nucleic acid having a base sequence complementary to the base sequence (antisense strand=non-coding strand) under high stringent conditions can be preferably mentioned. Being “capable of hybridizing under high stringent conditions” means as defined above. The nucleic acid may be a DNA or RNA, or a DNA/RNA chimera. Preferably, DNA can be mentioned.

The nucleic acid to be used as a probe may be double strand or single strand. In the case of a double-strand polynucleotide, it may be a double-strand DNA, a double-strand RNA or a DNA:RNA hybrid. In the case of a single strand, a sense strand (e.g., in case of cDNA, cRNA) or an antisense strand (e.g., in case of mRNA, cDNA) can be selected for use according to the sample to be used. The length of the nucleic acid is not particularly limited as long as it can specifically hybridize to a target nucleic acid and, for example, it is not less than about 15 bases, preferably not less than about 30 bases. The nucleic acid is preferably labeled with a label to enable detection or quantitation of a target nucleic acid.

As examples of the labeling agent, a radioisotope, an enzyme, a fluorescent substance, a luminescent substance and the like can be used. As examples of the radioisotope, [³²P], [³H], [¹⁴C] and the like can be used. As the enzyme, those that are stable and high in specific activity are preferred; for example, β-galactosidase, β-glucosidase, alkaline phosphatase, peroxidase, malate dehydrogenase and the like can be used. As examples of the fluorescent substance, fluorescamine, fluorescein isothiocyanate and the like can be used. As examples of the luminescent substance, luminol, luminol derivative, luciferin, lucigenin and the like can be used. Furthermore, a biotin-(strepto)avidin system can also be used for binding of a probe and a labeling agent. For immobilization of a nucleic acid to be a probe on a solid phase, a nucleic acid in a sample can be labeled using a label similar to those mentioned above.

An oligonucleotide set to be used as a primer is not particularly limited as long as it can specifically hybridize to each of a base sequence shown in each SEQ ID NO (sense strand) and a base sequence complementary thereto (antisense strand), and amplify a DNA fragment sandwiched therebetween and, for example, a set of oligo DNAs each designed to have a length of about 15 to about 100 bases, preferably about 15 to about 50 bases, and amplify about 100 bp to several kbp DNA fragments can be mentioned.

For quantitative analysis of PLsis marker gene expression using a trace amount of an RNA sample, competitive RT-PCR or real-time RT-PCR is preferably used. Competitive RT-PCR is a method comprising calculating the amount of an object DNA by carrying out a competitive amplification reaction in a reaction mixture in the co-presence of a known amount of other template nucleic acid as a competitor, which can be amplified by a primer set capable of amplifying the object DNA, and comparing the amounts of the amplification products. When using competitive RT-PCR, therefore, the reagent of the present invention can further contain, besides the above-mentioned primer set, a nucleic acid which is amplified by the primer set to produce an amplification product distinguishable from the object DNA (e.g., amplification product different from the object DNA in size, amplification product showing different migration pattern by a restriction enzyme treatment and the like). This competitor nucleic acid may be a DNA or an RNA. In the case of a DNA, cDNA is synthesized from an RNA sample by a reverse transcription reaction and a competitor is added to perform PCR, and in the case of an RNA, it may be added to an RNA sample from the start to perform RT-PCR. In the latter case, an absolute amount of the original mRNA can also be assumed since the efficiency of the reverse transcription reaction is taken into consideration.

On the other hand, real-time RT-PCR does not require electrophoresis, since the amplification amount by PCR can be monitored real-time, and the expression of PLsis marker gene can be analyzed more rapidly. Generally, monitoring is performed using various fluorescent reagents. These include reagents (intercalator) emitting fluorescence by binding to double stranded DNA such as SYBR Green I, ethidium bromide and the like, nucleic acids usable as the above-mentioned probes (the nucleic acid hybridizes to the target nucleic acid within amplification region), wherein the both ends are respectively modified with a fluorescent substance (e.g., FAM, HEX, TET, FITC etc.) and a quenching substance (e.g., TAMRA, DABCYL etc.) and the like.

The nucleic acid functionable as a probe capable of detecting the expression of a PLsis marker gene can be obtained by amplifying a nucleic acid having a desired length by PCR using the above-mentioned primer set capable of amplifying a part or whole of a transcription product of the gene and using cDNA or genomic DNA of any cell (for example, hepatocyte, splenocyte, nerve cell, glial cell, pancreatic β cell, myelocyte, mesangial cell, Langerhans' cell, epidermal cell, epithelial cell, goblet cell, endothelial cell, smooth muscle cell, fibroblast, fibrocyte, myocyte, adipocyte, immune cell (e.g., macrophage, T cell, B cell, natural killer cell, mast cell, neutrophil, basophil, eosinophil, monocyte), megakaryocyte, synovial cell, chondrocyte, bone cell, osteoblast, osteoclast, mammary gland cell, hepatocyte or interstitial cell, or corresponding precursor cell, stem cell or cancer cell thereof, and the like) of a mammal (for example, human, monkey, bovine, horse, swine, sheep, goat, dog, cat, rabbit, hamster, guinea pig, mouse, rat and the like), or any tissue where such cells are present (for example, brain, any portion of the brain (for example, olfactory bulb, amygdaloid nucleus, basal ganglia, hippocampus, thalamus, hypothalamus, cerebral cortex, medulla oblongata, cerebellum), spinal cord, hypophysis, stomach, pancreas, kidney, liver, gonad, thyroid, gall-bladder, bone marrow, adrenal gland, skin, lung, gastrointestinal tract (e.g., large intestine, small intestine), blood vessel, heart, thymus, spleen, submandibular gland, peripheral blood, prostate, testicle, ovary, placenta, uterus, bone, joint, adipose tissue, skeletal muscle, and the like) as a template, or cloning the above-mentioned PLsis marker gene or cDNA from cDNA or genomic DNA library derived from the aforementioned cell or tissue by colony or plaque hybridization and the like and, where necessary, treating same to give a fragment having a suitable length with a restriction enzyme and the like. The hybridization can be carried out according to the method described in, for example, Molecular Cloning, 2nd. ed. (mentioned above) and the like. When a commercially available library is used, hybridization can be carried out according to the method described in the instruction manual attached to the library. Alternatively, the nucleic acid can also be obtained by chemically synthesizing a part or whole of the base sequence and/or its complementary strand sequence based on the information of the base sequences shown in SEQ ID NOs:1, 3, 5, 7, 9, 11, 13, 15, 17, 19, 21 and 23 using a commercially available DNA/RNA automatic synthesizer and the like. In addition, a chip with a solid phased nucleic acid can also be prepared by direct in situ (on chip) synthesis of the nucleic acid on a solid phase such as silicone, glass and the like.

The nucleic acid functionable as a primer capable of amplifying a part or whole of a transcription product of a PLsis marker gene can also be obtained by chemically synthesizing a part of a base sequence and its complementary strand sequence, based on the information of the base sequences shown in SEQ ID NOs:1, 3, 5, 7, 9, 11, 13, 15, 17, 19, 21 and 23, using a commercially available DNA/RNA automatic synthesizer and the like.

The nucleic acid capable of detecting the expression of a PLsis marker gene can be provided as a solid in a dry state or in the form of an alcohol precipitate, or in a dissolution state in water or a suitable buffer (e.g., TE buffer etc.). When used as a labeled probe, the nucleic acid can be provided after labeling with any of the above-mentioned labeling substances, or provided independently of a labeling substance and labeled when in use.

Alternatively, the nucleic acid can be immobilized on a suitable solid phase. The solid phase is exemplified by, but not limited to, glass, silicone, plastic, nitrocellulose, nylon, polyvinylidene difluoride and the like. The solidifying means is exemplified by, but not limited to, a method comprising previously introducing a functional group such as amino group, aldehyde group, SH group, biotin and the like into a nucleic acid, introducing, into a solid phase, a functional group (e.g., aldehyde group, amino group, SH group, streptavidin and the like) reactive with the nucleic acid, and crosslinking the solid phase and the nucleic acid with covalent bond between these functional groups, or immobilizing a polyanionic nucleic acid with electrostatic bond using a polycation coating solid phase and the like.

One preferable embodiment of a nucleic acid probe immobilized on a solid phase is ArrayPlate™ etc. provided by High Throughput Genomics, Inc. ArrayPlate™ is a 96 well plate immobilized with various nucleic acid probes regularly disposed in the bottom of each well (e.g., 4×4 array). By the presence of a nucleic acid having one end hybridizable to a probe and the other end hybridizable to a target nucleic acid as a mediating spacer, a hybridization reaction of the probe with a target nucleic acid can be carried out in a liquid phase rather than on the solid phase surface, which enables a quantitative measurement of the target nucleic acid. Accordingly, expression variation of various PLsis marker genes can be simultaneously detected at once in a single well, and once sufficient quantitation performance is achieved, an advantage of higher efficiency than real-time PCR wherein expression variation in each marker gene is individually detected can be afforded.

While the reagent of the present invention has been explained with the emphasis on a nucleic acid capable of detecting a gene having the same or substantially the same base sequence as a base sequence shown by any of SEQ ID NOs:1, 3, 5, 7, 9, 11, 13, 15, 17, 19, 21 and 23, the reagent of the present invention may contain a nucleic acid capable of detecting a PLsis marker gene other than the above-mentioned 12 PLsis marker genes, such as a gene encoding a lysosomal enzyme, a gene encoding a lipid metabolism (e.g., cholesterol synthesis, fatty acid elongation, unsaturated fatty acid synthesis etc.)-related protein, a gene encoding a transport (e.g., fatty acid transport, protein transport, amino acid transport etc.)-related protein, a gene encoding a cell growth-related protein, a gene encoding a protease or protease inhibitor, a gene encoding an amino acid metabolism-related protein and the like, more specifically, human genes having base sequences registered in the GenBank database under the ID Nos. of NM_{—}000859, AL518627, NM_{—}002130, AA639705, BC005807, AF116616, NM_{—}025225, D80010, NM_{—}001731, AW134535, NM_{—}004354, AF135266, AC007182, NM_{—}003832, NM_{—}019058, AB040875, AA488687, NM_{—}018687, NM_{—}021158, BG231932, NM_{—}000235, AA873600, AF096304, AW150953, NM_{—}001360, AC001305, NM_{—}024090, NM_{—}006214, NM_{—}024108, NM_{—}021980, AF003934, NM_{—}000596, U15979, M92934, NM_{—}002087, AK023348, NM_{—}002773, NM_{—}000131, BC003169, NM_{—}002217, NM_{—}003122, NM_{—}001673, NM_{—}000050, U08024, NM_{—}003167, BC005161, AF162690, AW517464, AF116616, NM_{—}017983, NM_{—}016061, BE966922, BE552428, NM_{—}012445, NM_{—}000792, NM_{—}015930, NM_{—}021800, NM_{—}005980, NM_{—}000565, AB033025, AL110298, NM_{—}006931, NM_{—}001955, NM_{—}003897, AA778684, NM_{—}001283, NM_{—}012242, AI934469, NM_{—}003186 and NM_{—}002450, homologues thereof in other mammals and the like.

The reagent of the present invention can further contain, in addition to a nucleic acid capable of detecting the expression of a PLsis marker gene, other substance necessary for detecting the expression of the gene, which does not adversely affect the reaction when preserved in coexistence. Alternatively, the reagent of the present invention can also be provided as a kit together with a separate reagent containing other substance necessary for the reaction for detecting the expression of a PLsis marker gene. For example, when the reaction for detecting the expression of a PLsis marker gene is PCR, as said other substance, for example, a reaction buffer, dNTPs, a heat resistance DNA polymerase and the like can be mentioned. When competitive PCR or real-time PCR is used, a competitor nucleic acid, a fluorescent reagent (the above-mentioned intercalator, fluorescence probe etc.) and the like can be further contained.

Individual PLsis marker genes are not such that they show expression variation for any PLsis-inducing compound and substantially no expression variation for any PLsis non-inducing compound. Therefore, when expression of each marker gene is used as a single index, emergence of a certain extent of false-positive and false-negative compounds is inevitable. However, by examining expression variation of plural PLsis marker genes, the prediction hitting ratio can be further improved.

Accordingly, the present invention also provides a kit for prediction of PLsis induction potential of a drug, which contains two or more reagents containing a nucleic acid capable of detecting a PLsis marker gene in combination. The nucleic acid to be contained in each reagent can detect PLsis marker genes different from each other. The PLsis marker gene to be detected is not particularly limited, and the above-mentioned ones can be mentioned as examples thereof. Preferably, at least one of the reagents contains a nucleic acid capable of detecting a gene having a base sequence the same or substantially the same as the base sequence shown by any of SEQ ID NOs:1, 3, 5, 7, 9, 11, 13, 15, 17, 19, 21 and 23. More preferably, a kit that detects any two or more, further preferably 3 or more, still more preferably 4 or more, particularly preferably 5 or more, and most preferably 6 or more, of the above-mentioned 12 PLsis marker genes can be mentioned.

The nucleic acid contained in each reagent constituting the kit is particularly preferably so constructed as to detect expression of a PLsis marker gene by the same method (e.g., northern blot, dot blot, DNA array technique, quantitative RT-PCR etc.).

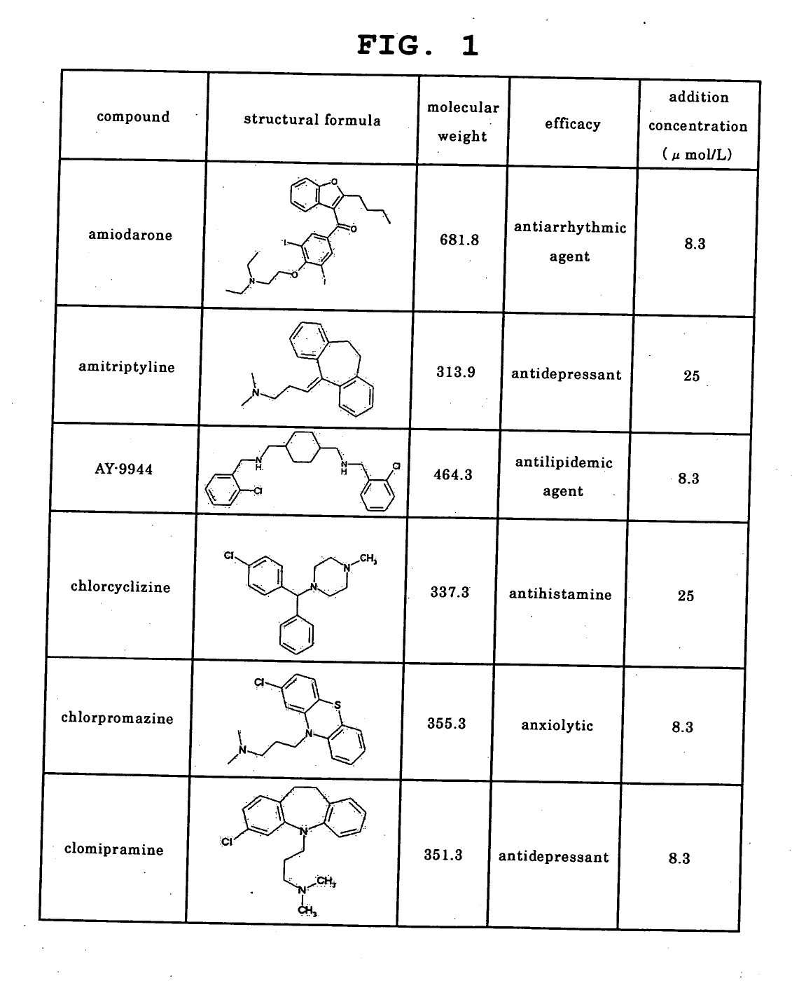

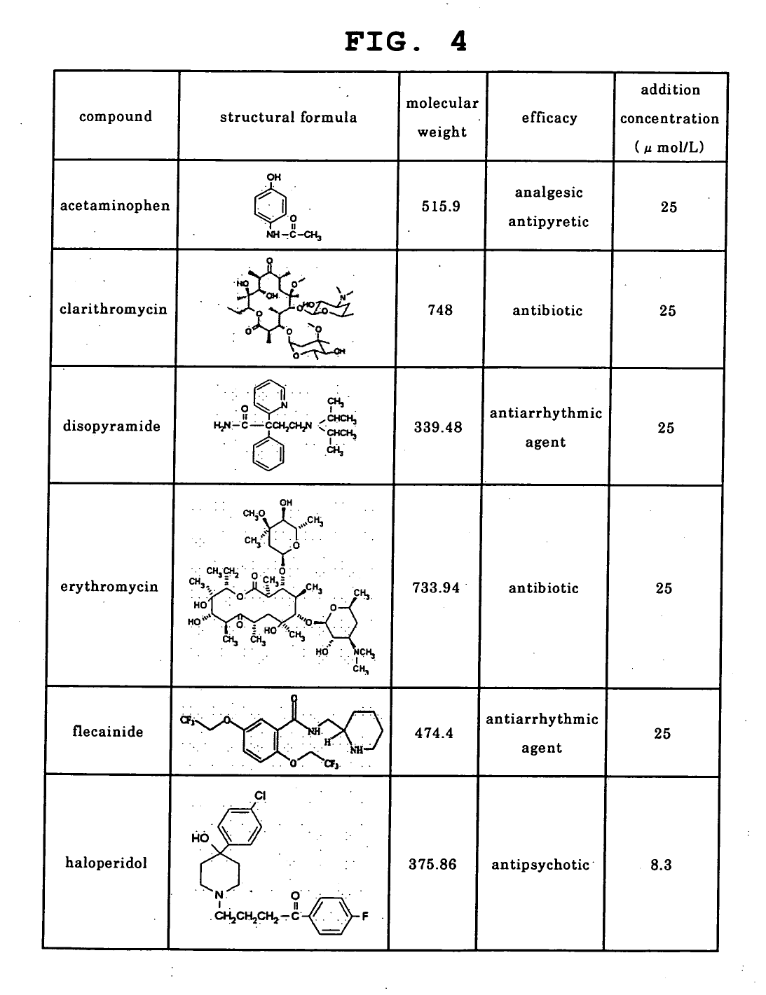

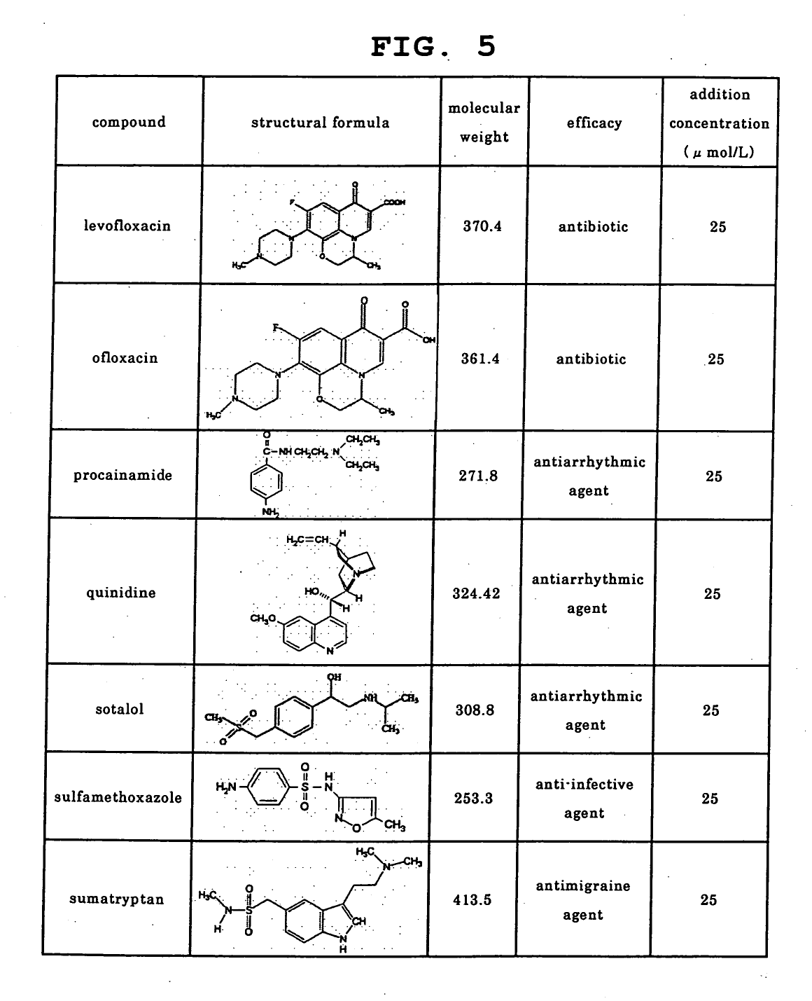

Alternatively, as a combination of preferable marker genes, a combination affording a prediction hitting ratio of PLsis induction potential of not less than about 70%, more preferably not less than about 80%, further preferably not less than about 90%, and particularly preferably not less than about 95%, using, as an index, an average variation rate (explained in detail in the explanation of the PLsis induction potential prediction method below) of expression upon exposure of mammalian cells to a test compound can be mentioned. As used herein, by the hitting of prediction is meant that, when a compound predicted to be PLsis positive is exposed to a mammalian cell, a myelin-like structure or a structure having a high electron density, which is an early image thereof, is observed in the cell, and when a compound predicted to be PLsis negative is exposed to a mammalian cell, a myelin-like structure or a structure having a high electron density, which is an early image thereof, is not observed in the cell. In this specification, the prediction hitting ratio is calculated with the PLsis positive compounds described in FIGS. 1-3 and the PLsis negative compounds described in FIGS. 4-5 as standard compounds.

The constitution of the kit of the present invention is exemplified by, but not limited to, one wherein the above-mentioned reagents of the present invention are separately provided [e.g., when nucleic acid functions as a labeled probe (particularly dot blot analysis), a primer for PCR (particularly real-time quantitative PCR) etc.], one wherein nucleic acids capable of detecting the expression of different PLsis marker genes are contained in a single reagent [e.g., when nucleic acid functions as PCR (particularly, each marker gene can be distinguished by the size of an amplification product and the like), a labeled probe (particularly, each marker gene can be distinguished by the size of a transcription product by northern blot analysis) and the like], one wherein nucleic acids capable of detecting the expression of different PLsis marker genes are immobilized in separate regions of a single solid phase [e.g., when functions as a probe for hybridization to label cRNA etc., and the like] and the like.

The present invention also provides a method for predicting a PLsis induction potential of a compound, which comprises detecting expression variation of one or more PLsis marker genes when a test compound is exposed to a mammalian cell-containing sample or a human or non-human mammal.

As the compound to be tested by the method of the present invention, for example, candidate compounds of pharmaceutical agent or animal drug and the like can be mentioned. Particularly, application to many candidate compound groups synthesized in the initial stage of drug discovery is preferable, since many samples can be treated rapidly. In this case, cell-containing samples or non-human mammals are used as test subject. On the other hand, expression variation of a PLsis marker gene can be preferably used in the final phase of development of pharmaceutical products (clinical trial), since it can be determined using a cell-containing sample that can be easily obtained, such as blood and the like.

As a mammalian cell-containing sample, any cell of a mammal (for example, human, monkey, bovine, horse, swine, sheep, goat, dog, cat, rabbit, hamster, guinea pig, mouse, rat and the like), desirably a mammal to be the administration subject of a test compound (e.g., hepatocyte, splenocyte, nerve cell, glial cell, pancreatic β cell, myelocyte, mesangial cell, Langerhans' cell, epidermal cell, epithelial cell, goblet cell, endothelial cell, smooth muscle cell, fibroblast, fibrocyte, myocyte, adipocyte, immune cell (e.g., macrophage, T cell, B cell, natural killer cell, mast cell, neutrophil, basophil, eosinophil, monocyte), megakaryocyte, synovial cell, chondrocyte, bone cell, osteoblast, osteoclast, mammary gland cell, interstitial cell, or corresponding precursor cell, stem cell or cancer cell thereof, and the like), or any tissue where such cells are present (for example, brain, any portion of the brain (for example, olfactory bulb, amygdaloid nucleus, basal ganglia, hippocampus, thalamus, hypothalamus, cerebral cortex, medulla oblongata, cerebellum), spinal cord, hypophysis, stomach, pancreas, kidney, liver, gonad, thyroid, gall-bladder, bone marrow, adrenal gland, skin, lung, gastrointestinal tract (e.g., large intestine, small intestine), blood vessel, heart, thymus, spleen, submandibular gland, peripheral blood, prostate, testicle, ovary, placenta, uterus, bone, joint, adipose tissue, skeletal muscle, and the like), a cell line established from the above-mentioned cell or tissue and the like can be mentioned. Preferably, hepatocyte, renal cells, monocyte, peripheral blood lymphocyte, fibroblast, adrenal gland steroid-producing cells, testis cell, ovary cell, abdominal cavity macrophage, alveolar epithelial cell, bronchial epithelial cell, alveolar macrophage and the like can be mentioned. In addition, use of a cell line is preferable in view of superior reproducibility (particularly in the case of human cell), easy availability and the like. As a human cell line, for example, liver cancer-derived HepG2 cell line, lymphoma-derived U-937 cell line, monocyte-derived THP-1 cell line, colorectal cancer-derived Caco-2 cell line, cervical cancer-derived HeLa cell line and the like can be mentioned.

The non-human mammal is exemplified by, but not limited to, rat, hamster, guinea pig, rabbit, mouse, monkey, dog, swine, cat, sheep, goat, horse, bovine and the like. Preferred are rat, hamster, guinea pig, rabbit, mouse, monkey, dog and the like.

While a method for exposing a mammalian cell-containing sample to a test compound is not particularly limited, specifically when, for example, a cell line is used as a sample, cells in a cell growth phase, which were cultured in a suitable medium under preferable conditions are detached using trypsin-EDTA and the like, recovered by centrifugation, and a suitable medium [e.g., MEM medium (Science, 122: 501 (1952)), DMEM medium (Virology, 8: 396 (1959)), RPMI 1640 medium (The Journal of the American Medical Association, 199:519 (1967)), 199 medium (Proceeding of the Society for the Biological Medicine, 73: 1 (1950)) containing about 5 to about 20% fetal bovine serum (FBS) and the like (where necessary, antibiotics such as penicillin, streptomycin, hygromycin and the like may be further added)] is added to give a suspension at a desired cell density. While the cell density is not particularly limited as long as the gene expression and its variation can be detected, it is preferable to so control the cells as to keep the cell growth phase. Therefore, preferable initial cell density varies depending on the growth rate of the cells to be used and the like, which can be easily determined by those of ordinary skill in the art according to the cells to be used. It is generally about 5×10⁴- about 1×10⁷ cells/mL. A test compound dissolved in a suitable solvent is further diluted with the medium, added to the above-mentioned cell suspension to a final concentration of, for example, the highest concentration at which the cells can survive (the concentration can be determined separately by histological observation), and cultivated under conventional conditions, for example, in a CO₂ incubator under an atmosphere of 5% CO₂/95% air, 5% CO₂/5% O₂/90% air and the like, at about 30° C.- about 40° C. for about 0.5- about 168 hr, preferably about 3- about 48 hr, more preferably about 23- about 25 hr.

A method for exposing a mammal to a test compound is not particularly limited as long as it includes administration of the test compound to the animal such that a sufficient amount of the test compound reaches the target cell (cell contained in a sample to be recovered later from the animal to examine expression variation of a PLsis marker gene) and, for example, the test compound can be administered in the form of a solid, a semi-solid, a liquid, an aerosol and the like orally or parenterally (e.g., intravenously, intramuscularly, intraperitoneally, intraarterially, subcutaneously, intradermally, airway etc.). The dose of the test compound varies depending on the kind of the compound, animal species, body weight, administration form and the like and, for example, an amount within the range where the animal can survive and necessary for the target cell to be exposed to the highest concentration of the test compound, at which the cell can survive, for more than a given time and the like can be mentioned. The dose can be administered in one to several portions. While the time from the administration to the sample collection varies depending on the in vivo kinetics of the test compound and the like, it is generally about 3 hr to about 3 days from the initial administration.

As a sample to be taken from a mammal administered with the test compound, those containing various cells exemplified as the mammalian cell-containing sample can be preferably mentioned. Blood (e.g., peripheral blood) and the like are particularly preferable since they can be recovered rapidly and conveniently, invasion into an animal is few and the like.

The PLsis marker gene whose expression variation is examined in the prediction method of the present invention is not particularly limited and, for example, a gene encoding a lysosomal enzyme, a gene encoding a lipid metabolism (e.g., cholesterol synthesis, fatty acid elongation, unsaturated fatty acid synthesis etc.)-related protein, a gene encoding a transport (e.g., fatty acid transport, protein transport, amino acid transport etc.)-related protein, a gene encoding a cell growth-related protein, a gene encoding a protease or protease inhibitor, a gene encoding an amino acid metabolism-related protein and the like can be mentioned. More specifically, as a gene that shows an increased expression in correlation with PLsis, human genes having base sequences registered in the GenBank database under the ID Nos. of NM_{—}014960, NM_{—}000859, AL518627, NM_{—}002130, AA639705, BC005807, AF116616, NM_{—}025225, U47674, D80010, NM_{—}001731, AW134535, NM_{—}004354, AF135266, AC007182, NM_{—}003832, NM_{—}019058, AB040875, AA488687, NM_{—}018687, NM_{—}021158, BG231932, NM_{—}024307, NM_{—}000235, AA873600, D63807, AF096304, AW150953, NM_{—}001360, NM_{—}021969, AC001305, NM_{—}024090, NM_{—}001443, NM_{—}006214, NM_{—}024108, NM_{—}021980, NM_{—}002151, AF003934, NM_{—}000596, U15979, M92934, NM_{—}002087, AK023348, NM_{—}002773, NM_{—}000131, BC003169, NM_{—}002217, NM_{—}003122, NM_{—}001673, NM_{—}000050, NM_{—}001085, U08024, NM_{—}003167, BC005161, AF162690, AW517464, AF116616, NM_{—}017983, AL136653, NM_{—}016061, BE966922, BE552428, NM_{—}022823, NM_{—}012445, NM_{—}000792, NM_{—}015930, NM_{—}021800, NM_{—}005980, NM_{—}000565 and AB033025, homologues thereof in other mammals and the like can be mentioned. On the other hand, as a gene that shows a decreased expression in correlation with PLsis, human genes having base sequences registered in the GenBank database under the ID Nos. of NM_{—}006931, AL110298, NM_{—}006931, NM_{—}001955, NM_{—}003897, NM_{—}003186, AA778684, NM_{—}001283, NM_{—}012242, AI934469, NM_{—}003186 and NM_{—}002450, homologues thereof in other mammals and the like can be mentioned.

Preferably, at least one of the PLsis marker genes capable of examining the expression variation of the prediction method of the present invention contains a base sequence the same or substantially the same as the base sequence shown by any of SEQ ID NOs:1, 3, 5, 7, 9, 11, 13, 15, 17, 19, 21 and 23. More preferably, a method that detects any two or more, further preferably 3 or more, still more preferably 4 or more, particularly preferably 5 or more, and most preferably 6 or more, of the above-mentioned 12 PLsis marker genes can be mentioned.

Alternatively, as a combination of preferable marker genes, a combination affording a prediction hitting ratio of PLsis induction potential of not less than about 70%, more preferably not less than about 80%, further preferably not less than about 90%, and particularly preferably not less than about 95%, using, as an index, an average variation rate of expression of all marker genes by the present prediction method can be mentioned. In the prediction method of the present invention, the “average variation rate” is defined as follows. That is, an expression amount is measured for each marker gene when mammal (cells) is and is not exposed to a test compound and when the expression amount increased upon exposure, its magnification (e.g., 2 when increased two-fold) is taken as an expression variation rate (X) of each gene, and when the expression amount decreased, an inverse number of its magnification (e.g., 2 when decreased to ½) is taken as an expression variation rate (X) of each gene, and an average value of the expression variation rate of the total marker genes (n genes) is defined to be an average variation rate (following formula).
Average variation rate=m₁X₁+m₂X₂+ . . . +mₙXₙ(m₁+m₂+ . . . +mₙ=1)
wherein mᵢ (i=1−n) shows the weight of each gene. While the weight is not particularly limited, it is preferably mᵢ×n=0.2−5, for example, it is the same weight for all (mᵢ=1/n).

In the method of the present invention, when the average variation rate is not less than the standard value determined by the below-mentioned method, PLsis positive is predicted and when it is less than the standard value, PLsis negative is predicted.

The expression of a PLsis marker gene in a sample containing a mammalian cell exposed to the test compound and a sample taken from a mammal administered with the test compound can be examined by preparing an RNA (e.g., total RNA, mRNA) fraction from the sample, and detecting a transcription product of the marker gene contained in the fraction. An RNA fraction can be prepared by a known means such as guanidine-CsCl ultracentrifugation method, AGPC method and the like. A high purity total RNA can be prepared rapidly and conveniently from a trace sample using a commercially available RNA extraction kit (e.g., RNeasy Mini Kit; manufactured by QIAGEN etc.). As a means for detecting a transcription product of a PLsis marker gene in an RNA fraction, for example, a method using hybridization (northern blot, dot blot, DNA chip analysis etc.), a method using PCR (RT-PCR, competitive PCR, real-time PCR etc.) and the like can be mentioned. Since expression variation of a PLsis marker gene can be detected rapidly and conveniently with high quantitation performance from a trace sample, quantitative PCR such as competitive PCR, real-time PCR and the like, and since expression variation of multiple marker genes can be detected at once and quantitation performance can be improved by the selection of a detection method and the like, DNA chip analysis is preferable.

In the case of northern blot or dot blot hybridization, a PLsis marker gene can be detected using the above-mentioned reagent or kit of the present invention containing a nucleic acid to be used as a labeled probe. In the case of Northern hybridization, therefore, an RNA fraction prepared as mentioned above is separated by gel electrophoresis, transferred to a membrane such as nitrocellulose, nylon, polyvinylidene difluoride and the like, hybridized under the above-mentioned “high stringent conditions” in a hybridization buffer containing the reagent of the present invention or each reagent contained in the kit of the present invention, the amount of a label bonded to the membrane by a suitable method is measured for each band, whereby the expression amount of each PLsis marker gene can be measured. In the case of dot blot, too, a membrane spotted with an RNA fraction is subjected to a hybridization reaction in the same manner (conducted for each PLsis marker gene), and the label amount of the spot is measured, whereby the expression amount of each marker gene can be measured.

In the case of DNA chip analysis (solid phased probe described for the above-mentioned reagent of the present invention), for example, cDNA incorporating a suitable promoter such as T7 promoter and the like is synthesized by reverse transcription reaction from the RNA fraction prepared as mentioned above, and cRNA is synthesized using an RNA polymerase (where labeled cRNA is obtained by using, as a substrate, a mononucleotide labeled with biotin and the like). The labeled cRNA is contacted with the above-mentioned solid phased probe to allow hybridization reaction, and a label amount bonded to each probe on the solid phase is measured, whereby the expression amount of each PLsis marker can be measured. This method is advantageous in terms of rapidness and convenience as the number of PLsis marker genes (i.e., probes to be solid phased) to be detected increases.

In a preferable embodiment of the prediction method of the present invention, as a method for detecting the expression of a PLsis marker gene, quantitative PCR is used. As quantitative PCR, for example, competitive PCR, real-time PCR and the like can be mentioned. Since electrophoresis after amplification reaction is not necessary, real-time PCR is more superior in rapid performance.

In the case of competitive PCR, a known amount of a competitor nucleic acid is used, which can be amplified by said primer set in addition to the primer set described for the above-mentioned reagent of the present invention, and which, after amplification, can be distinguished from an amplification product of a target nucleic acid (i.e., a transcription product of a PLsis marker gene), based on different amplification size, different migration pattern of restriction enzyme-treated fragment and the like. Since amplification occurs competitively where a target nucleic acid and a competitor nucleic acid compete for a primer, the amount ratio of the amplification products reflects the amount ratio of the original templates. The competitor nucleic acid may be DNA or RNA. In the case of DNA, cDNA is synthesized from an RNA fraction prepared as mentioned above by reverse transcription reaction, and then PCR is performed in the coexistence of the reagent of the present invention and a competitor, and in the case of RNA, a competitor is added to an RNA fraction to allow reverse transcription reaction, and the reagent of the present invention is further added to perform PCR.

In real-time PCR, the amplification amount is monitored in real-time using a fluorescent reagent, and an apparatus integrally comprising a thermal cycler and a spectrofluorophotometer is necessary. Such apparatus is commercially available. There are several methods depending on the fluorescent reagent to be used and, for example, intercalator method, TaqMan™ probe method, Molecular Beacon method and the like can be mentioned. In any case, cDNA is synthesized by reverse transcription reaction from an RNA fraction prepared as mentioned above, and the reagent of the present invention and a fluorescent reagent (probe) called intercalator, TaqMan™ probe or Molecular Beacon probe is added to each PCR reaction system. Since intercalator binds to a synthesized double stranded DNA and emits fluorescence upon irradiation of excitation light, the amount of an amplification product can be monitored by measuring the intensity of fluorescence, based on which the amount of original template cDNA can be assumed. The TaqMan™ probe is an oligonucleotide capable of hybridizing to an amplification region of the target nucleic acid, which has both ends modified by a fluorescent substance and a quenching substance, respectively. It hybridizes to a target nucleic acid during annealing but is prohibited from emitting fluorescence by the presence of the quenching substance, and emits fluorescence when decomposed by the exonuclease activity of DNA polymerase during elongation, which liberates the fluorescent substance. Accordingly, by measuring fluorescence intensity, the amount of the amplification product can be monitored, based on which the amount of original template cDNA can be assumed. The Molecular Beacon probe is an oligonucleotide capable of hybridizing to an amplification region of a target nucleic acid and having a hairpin type secondary structure, which has both ends modified by a fluorescent substance and a quenching substance, respectively. When it has a hairpin structure, it does not emit fluorescence due to the presence of a quenching substance, and emits fluorescence when the distance between the fluorescent substance and the quenching substance grows upon hybridization to the target nucleic acid during annealing. Therefore, the amount of the amplification product can be monitored by measuring the fluorescence intensity, based on which the amount of original template cDNA can be assumed.

In the prediction method of the present invention, the standard according to which the presence or absence of a PLsis induction potential of a compound is judged is not particularly limited as long as the prediction results based on the standard have sufficient reliability for use as a compound screening system. For example, (1) a method for judging PLsis positive when expression of all PLsis marker genes to be the detection target substantially increases or decreases (where “expression substantially increases or decreases” means as defined above) due to exposure to the test compound, and judging PLsis negative when expression of any of the PLsis marker genes does not substantially change (where “expression does not substantially change” means as defined above) due to exposure to the test compound, (2) a method for judging PLsis negative when expression of all PLsis marker genes to be the detection target does not substantially change due to exposure to the test compound, and judging PLsis positive when expression of any of PLsis marker genes substantially increases or decreases due to exposure to the test compound, (3) a method for judging PLsis positive when expression of not less than a certain number (e.g., 2-(n−1) genes) out of PLsis marker genes in the number of n to be the detection target substantially increases or decreases due to exposure to the test compound and the like can be mentioned. However, according to the above-mentioned method (1), the frequency of appearance of false-positive compounds can be reduced, but the frequency of appearance of false-negative compounds increases and a considerable number of PLsis-inducing compounds cannot be eliminated. On the other hand, according to the method of (2), the frequency of appearance of false-negative compounds can be reduced, but the frequency of appearance of false-positive compounds increases, which possibly eliminates potential compounds and the room of development of pharmaceutical products and the like may be narrowed.

The present invention provides a method for determining the judgment standard for maximally improving the reliability (prediction hitting ratio) system for the combination of selected PLsis marker genes. That is, this method comprises exposing mammalian cell-containing samples or humans or non-human mammals to each of not less than 2 (preferably not less than 5, more preferably not less than 10, further preferably not less than 15) known PLsis-inducing compounds (e.g., compounds described in FIGS. 1-3) and not less than 2 (preferably not less than 5, more preferably not less than 10, further preferably not less than 15) known PLsis non-inducing compounds (e.g., compounds described in FIGS. 4-5), detecting expression variation of one or more selected PLsis marker genes in the samples or samples taken from the mammals, and comparing the average expression variation rate of the marker genes (here, the “average variation rate” is as defined above) with the presence or absence of actual PLsis induction potential. In the present invention, the presence or absence of actual PLsis induction potential is determined by the appearance of a myelin structure or a structure having a high electron density, which is an early image thereof, in the cell on exposure of mammalian cell to a compound.

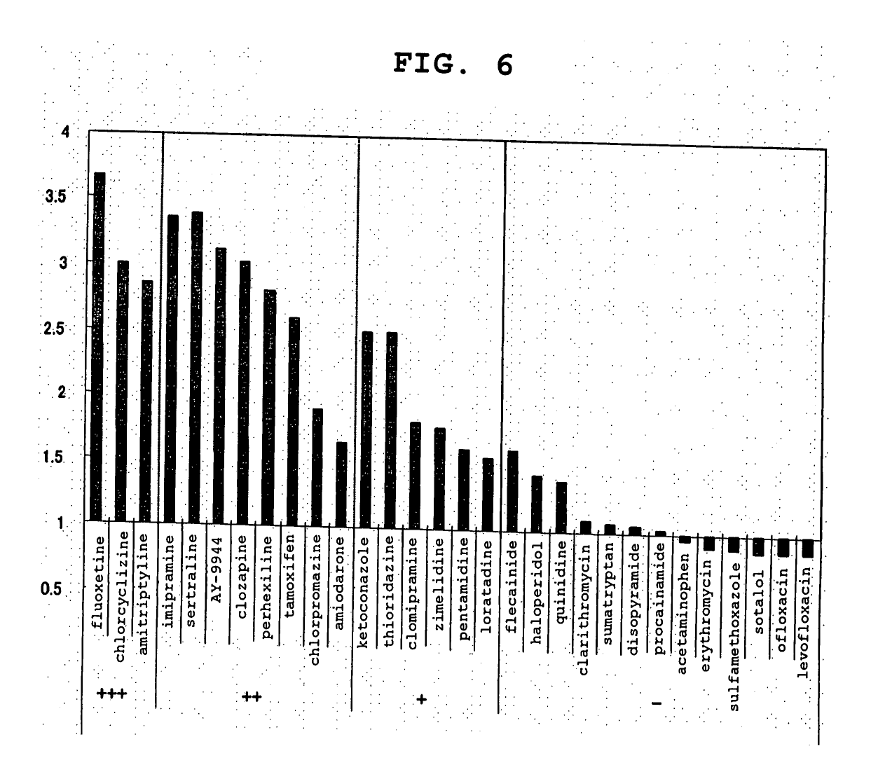

As a result of comparison, an average variation rate capable of correctly judging the presence or absence of the PLsis inducing property of the above-mentioned known PLsis inducing and non-inducing compounds with the probability of not less than about 70%, preferably not less than about 80%, more preferably not less than about 90%, particularly preferably not less than about 95%, is determined and used as a standard value. For example, when expression variation of 12 PLsis marker genes having the base sequences shown in SEQ ID NOs:1, 3, 5, 7, 9, 11, 13, 15, 17, 19, 21 and 23, respectively, is examined with regard to the compounds described in FIGS. 1-5, an average variation rate (weight of each gene is the same) is as shown in FIG. 6, and an average variation rate of 1.5 that can evaluate all of 17 kinds of PLsis-inducing compounds as positive and 12 kinds out of 13 kinds of PLsis non-inducing compounds as negative (therefore, evaluation with about 97% probability of correctness) can be determined as the standard value.

The standard value determined as mentioned above is more preferably examined for its validity by further comparing an average variation rate of a PLsis marker gene expression and the presence or absence of actual PLsis induction potential in a similar manner using known PLsis inducing and non-inducing compounds. When an average variation rate capable of correctly evaluating the presence or absence of a PLsis induction potential more accurately with higher probability can be obtained from overall evaluation results of newly examined compounds, the standard value only needs to be amended. Moreover, by accumulating the data relating to known compounds by evaluation by the present method and microscopic observation, extremely accurate prediction of compound groups unknown as to the presence or absence of a PLsis induction potential becomes possible.

The concept of the average variation rate to be preferably used as an index of the PLsis prediction method of the present invention can be applied to other toxicity prediction methods of compounds using an exhaustive gene expression analysis. For example, as a prediction method of hepatotoxicity (e.g., inducibility such as hepatitis, liver necrosis, fatty liver and the like), a screening system capable of predicting hepatotoxicity of a test compound with precision can be constructed by exposing hepatocytes to several kinds to several dozen kinds of known hepatotoxic compounds (e.g., acetaminophen, amitriptyline, ANIT, carbon tetrachloride, cyproterone acetate, estradiol, indomethacin etc.), extracting RNA, synthesizing cDNA and then labeled cRNA by a conventional method, fragmenting them, performing an exhaustive gene expression analysis using a commercially available mammal genome DNA microarray (e.g., GeneChip (trademark) manufactured by Affymetrix and the like), identifying, for example, gene groups wherein not less than half of the compounds examined showed common variation of expression as hepatotoxic marker genes, selecting some from these marker genes, and examining the average variation rate of marker gene expression upon exposure of the hepatocytes to the test compound in the same manner as above.

Abbreviations for bases, amino acids and the like used herein are based on abbreviations specified by the IUPAC-IUB Commission on Biochemical Nomenclature or abbreviations in common use in relevant fields. Some examples are given below. When an enantiomer may be present in amino acid, it is of the L-configuration, unless otherwise stated.
DNA: Deoxyribonucleic acid
cDNA: Complementary deoxyribonucleic acid
A: Adenine
T: Thymine
G: Guanine
C: Cytosine
RNA: Ribonucleic acid
mRNA: Messenger ribonucleic acid
dATP: Deoxyadenosine triphosphate
dTTP: Deoxythymidine triphosphate
dGTP: Deoxyguanosine triphosphate
dCTP: Deoxycytidine triphosphate
ATP: Adenosine triphosphate
EDTA: Ethylenediaminetetraacetic acid
SDS: Sodium dodecyl sulfate
Gly: Glycine
Ala: Alanine
Val: Valine
Leu: Leucine
Ile: Isoleucine
Ser: Serine
Thr: Threonine
Cys: Cysteine
Met: Methionine
Glu: Glutamic acid
Asp: Aspartic acid
Lys: Lysine
Arg: Arginine
His: Histidine
Phe: Phenylalanine
Tyr: Tyrosine
Trp: Tryptophan
Pro: Proline
Asn: Asparagine
Gln: Glutamine
pGlu: Pyrroglutamic acid
Sec: Selenocysteine

### EXAMPLES

The present invention is explained in more detail in the following by referring to Examples, which are mere examples and do not limit the scope of the present invention in any way.

### Example 1

### Reference Example

Electromicroscopic Examination of PLsis Induction Potential of Compound

Using 30 kinds of the following commercially available drugs as test compounds, the level of PLsis induction potential was examined with the emergence of an intracellular myelin-like structure or a structure having a high electron density, which is an early image thereof, as an index by electronmicroscopic observation. Amiodarone and clozapine were purchased from ICN Biomedicals, imipramine, clarithromycin, disopyramide, erythromycin, haloperidol, ketoconazole, quinidine, sertraline and sulfamethoxazole were purchased from Wako Pure Chemical Industries, Ltd., amitriptyline, AY-9944, chlorcyclizine, chlorpromazine, clomipramine, fluoxetine, perhexiline, tamoxifen, thioridazine, zimelidine, acetaminophen, flecainide, ofloxacin and sotalol were purchased from Sigma, levofloxacin was purchased from Apin Chemicals, loratadine and sumatriptan were purchased from KEMPROTEC, pentamidine was purchased from Tronto Research Chemicals and procainamide was purchased from Aldrich Chemical.

The test compound was dissolved in dimethylsulfoxide (DMSO) to the final concentration (separately, cells were exposed to the compound for 72 hr and the highest concentration at which the cells were viable was employed) of 8.3-25 μmol/L. The structural formulas, molecular weights, efficacy, addition concentrations of the test compounds are shown in FIGS. 1-5.

HepG2 cells (purchased from ATCC) were exposed to the test compound by a conventional method. HepG2 cells used were of the cell growth phase. The attached HepG2 cells were washed twice with. Dulbecco's phosphate buffered saline (calcium and magnesium salt free; PBS(−))(Dainippon Pharmaceutical Co., Ltd.) containing 0.05 w/v % EDTA, detached with a cell dissociation solution of 0.25 vol % trypsin-1 mmol/L EDTA (Gibco BRL) diluted 2-fold with PBS(−), centrifuged to remove the supernatant, and adjusted to a concentration of 2×10⁵ cells/250 μL with a culture medium [Dulbecco's modified Eagle medium (DMEM)(Gibco BRL) supplemented with 50 U/mL penicillin (Gibco BRL)-50 mmol/L streptomycin (Gibco BRL) and 5 vol % FBS (Bio whittaker)]. DMSO alone or a culture medium containing the above-mentioned test compound in DMSO solution was dispensed to a well by 250 μL, the above-mentioned cell suspension (250 μL) was added, and cultured in a CO₂ incubator (7100; Napco) under an atmosphere of 5% CO₂—95% air at 37° C.

After culture for 72 hr, the culture medium was removed, and the cells were fixed with 1 w/v % glutaraldehyde solution. After fixing with 2 w/v % osmic acid for 2 hr by a conventional method and dehydrating with an alcohol series, the cells were embedded in a resin (Quetol 812). Ultrathin slices were prepared and, after electron staining, observed with an electron microscope (H-300; Hitachi) and photographed in 3 or more pictures per each sample (magnification ×5000). The processing after post-fixation was performed by Applied Medical Research. The electron micrographs were grossly observed and the level of appearance of a myelin-like structure was divided into 4 classes of heavy, moderate, light, no change by a blind test (n=4). In the classification criteria, “heavy” means multiple large myelin-like structures, “medium” means a small number of moderate level myelin-like structures, “light” means a small number of minor myelin-like structures, and “no change” means absence of a myelin-like structure.

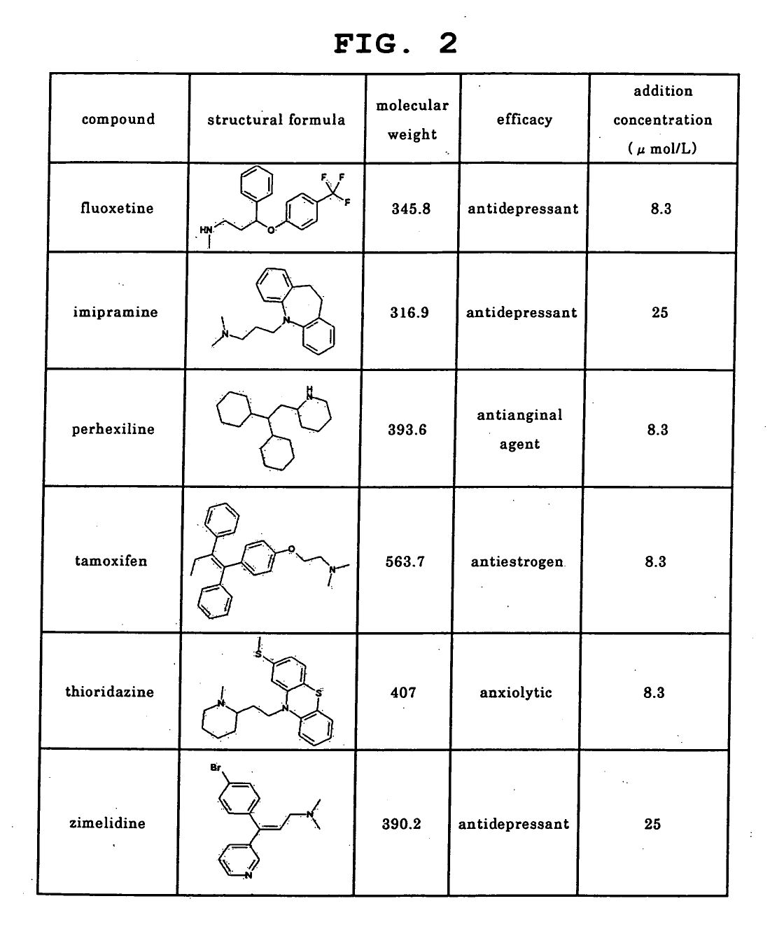

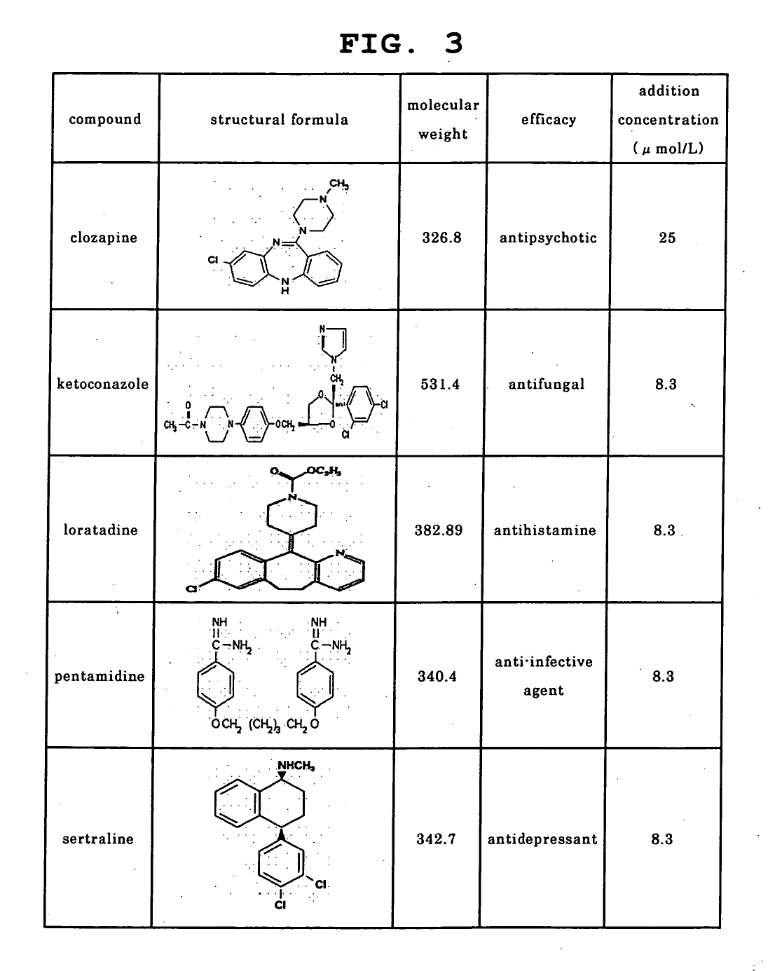

As a result, all of 12 known PLsis-inducing compounds (compounds shown in FIGS. 1 and 2), and 5 compounds out of 18 compounds for examination of evaluation system (compounds shown in FIG. 3) showed a myelin-like structure, which is a typical PLsis image, in lysosome. In contrast, 13 compounds (compounds shown in FIGS. 4 and 5) out of the compounds for examination of evaluation system showed no change in lysosome. The ranking of myelin-like structure appearance level is shown in Table 1 (addition concentration of compound shows the highest concentration of cells that survived after 72 hr of culture).

TABLE 1

| frequency of | | |
|---|---|---|
| appearance of | | addition |
| myelin-like | | concentration |
| structure | compound | (μmol/L) |
| severe | amitriptyline | 25 |
| | chlorcyclizine | 25 |
| | fluoxetine | 8.3 |
| moderate | amiodarone | 8.3 |
| | AY-9944 | 8.3 |
| | chlorpromazine | 8.3 |
| | imipramine | 25 |
| | tamoxifen | 8.3 |
| | perhexiline | 8.3 |
| | clozapine | 25 |
| | sertraline | 8.3 |
| mild | clomipramine | 8.3 |
| | thioridazine | 8.3 |
| | zimelidine | 25 |
| | ketoconazole | 8.3 |
| | loratadine | 8.3 |
| | pentamidine | 8.3 |
| no change | solvent | — |
| | acetaminophen | 25 |
| | clarithromycin | 25 |
| | disopyramide | 25 |
| | erythromycin | 25 |
| | flecainide | 25 |
| | haloperidol | 8.3 |
| | levofloxacin | 25 |
| | ofloxacin | 25 |
| | procainamide | 25 |
| | quinidine | 25 |
| | sotalol | 25 |
| | sulfamethoxazole | 25 |
| | sumatriptan | 25 |

### Example 2

Expression Variation of Various Genes Due to Exposure to Known PLsis Inducing and Non-Inducing Compounds

In the same manner as in Example 1 (Reference Example), HepG2 cells were exposed to 17 kinds of PLsis-inducing compounds and 14 kinds of PLsis non-inducing compounds for 24 hr each, the culture medium was removed and the cells were cryopreserved at −80° C. Using RNeasy Mini Kit (QIAGEN), total RNA was purified from the cells, and cDNA was synthesized in a 100 μL system using TaqMan Reverse Transcription Reagents (PE Applied Biosystems).

Based on the base sequences shown in SEQ ID NOs:1, 3, 5, 7, 9, 11, 13, 15, 17, 19, 21 and 23, primer and FAM labeled-probe were designed using Primer Express (PE Applied Biosystems) and synthesized (committed to SIGMA Genosys Japan).

The sequences of respective primers and probes are respectively shown in SEQ ID NOs:25-60. As the primer for glyceraldehyde-3-phosphoric acid dehydrogenase (GAPDH) and VIC labeled probe, those attached to TaqMan GAPDH Control Reagents (PE Applied Biosystems) were used.

Using 100 μL of the reaction mixture containing 5 μL of cDNA (1×TaqMan Universal PCR Master Mix (PE Applied Biosystems), 200 nM forward primer, 200 nM reverse primer and 200 nM TaqMan probe), 40 cycles of (1 cycle=95° C., 15 sec; 60° C., 1 min) PCR were performed. PCR and fluorescence detection were performed using ABI PRISM Sequence Detector 7000 (PE Applied Biosystems). As the internal standard, GAPDH was used to amend the measurement values. For the determination of significant difference, t-test was used (n=3).

For each test compound, the expression variation rate of 12 genes each relative to the control group was determined. The results are shown in Table 2. In all the 12 genes examined, there was a tendency toward variation in their expression due to the exposure to a PLsis-inducing compound, and substantially no variation in their expression due to the exposure to a PLsis non-inducing compound. Therefore, it has been clarified that these 12 genes are marker genes useful for the prediction of a PLsis induction potential of a drug.

TABLE 2

| electro- | frequency of appearance of myelin-like structure^{a} | |
|---|---|---|
| microscopic | +++ | ++ |

| test | | expression variation rate^{c} | | | | | | | | | | | |
|---|---|---|---|---|---|---|---|---|---|---|---|---|---|
| | gene^{b} | amitriptyline | | chlorcyclizine | | fluoxetine | | amiodarone | | AY-9944 | | chlorpromazine | |
| real-time | kiaa1001 | 2.11 | * | 2.47 | ** | 1.93 | ** | 1.56 | | 1.68 | * | 1.90 | * |
| quantitative | asah1 | 3.10 | * | 3.23 | * | 6.55 | * | 1.69 | | 4.71 | * | 1.29 | |
| PCR | mgc4171 | 2.22 | ** | 2.43 | ** | 1.34 | | 1.32 | | 1.63 | | 1.74 | * |
| | lss | 1.79 | ** | 2.98 | ** | 3.51 | | 1.82 | | 4.78 | * | 1.99 | ** |
| | nr0b2 | 1.64 | | 2.46 | * | 2.70 | | 1.35 | | 2.71 | ** | 1.20 | |
| | fabp1 | 2.39 | ** | 2.62 | ** | 2.80 | * | 2.01 | ** | 3.16 | ** | 2.56 | ** |
| | hpn | 2.20 | * | 2.97 | * | 2.39 | ** | 2.15 | ** | 2.68 | ** | 1.70 | ** |
| | serpina3 | 3.01 | ** | 2.23 | ** | 1.73 | ** | 1.17 | | 1.97 | * | 1.94 | ** |
| | depp | 5.08 | ** | 3.24 | * | 3.08 | * | 1.12 | | 3.32 | ** | 2.95 | ** |
| | flj22362 | 1.80 | ** | 2.96 | ** | 2.15 | ** | 1.07 | | 1.45 | | 1.70 | * |
| | slc2a3 | 0.22 | * | 0.20 | * | 0.09 | * | 0.35 | * | 0.22 | * | 0.43 | |
| | tagln | 0.23 | ** | 0.30 | ** | 0.20 | * | 0.56 | * | 0.21 | * | 0.65 | * |

| electro- | | frequency of appearance of myelin-like structure^{a} | | | | | | | | | | | |
|---|---|---|---|---|---|---|---|---|---|---|---|---|---|
| microscopic | | ++ | | | | | | | | | | + | |
| test | | expression variation rate^{c} | | | | | | | | | | | |
| | gene^{b} | imipramine | | perhexiline | | tamoxifen | | clozapine | | sertraline | | clomipramine | |
| real-time | kiaa1001 | 2.11 | ** | 1.98 | ** | 1.85 | ** | 3.34 | * | 2.38 | * | 3.83 | |
| quantitative | asah1 | 4.52 | | 3.12 | | 4.50 | ** | 2.75 | | 3.08 | | 0.82 | |
| PCR | mgc4171 | 1.81 | * | 1.93 | * | 1.60 | | 1.88 | | 1.94 | | 1.41 | |
| | lss | 2.69 | ** | 3.10 | ** | 3.01 | ** | 2.69 | ** | 3.91 | ** | 1.52 | * |
| | nr0b2 | 3.41 | * | 2.09 | * | 2.56 | ** | 1.97 | | 1.79 | | 0.98 | |
| | fabp1 | 2.45 | ** | 2.49 | ** | 1.90 | ** | 3.89 | ** | 4.14 | * | 2.36 | ** |
| | hpn | 2.32 | ** | 2.22 | * | 2.19 | * | 2.35 | ** | 3.07 | ** | 2.39 | ** |
| | serpina3 | 2.12 | ** | 2.50 | ** | 2.12 | ** | 1.96 | * | 2.69 | * | 2.03 | ** |
| | depp | 3.36 | ** | 2.55 | ** | 2.02 | | 5.15 | ** | 4.44 | * | 2.08 | |
| | flj22362 | 1.80 | * | 3.42 | ** | 1.53 | | 1.92 | ** | 3.49 | * | 1.46 | |
| | slc2a3 | 0.15 | * | 0.22 | * | 0.24 | * | 0.24 | ** | 0.14 | * | 0.63 | |
| | tagln | 0.15 | * | 0.26 | ** | 0.27 | * | 0.24 | ** | 0.37 | ** | 0.78 | |

| | | | | | | | | | | | | frequency of | |
|---|---|---|---|---|---|---|---|---|---|---|---|---|---|
| | | | | | | | | | | | | appearance of | |
| | | | | | | | | | | | | myelin-like | |
| | | | | | | | | | | | | structure | |
| electro- | | frequency of appearance of myelin-like structure^{a} | | | | | | | | | | − | |
| microscopic | | + | | | | | | | | | | expression | |
| test | | expression variation rate^{c} | | | | | | | | | | variation rate | |
| | gene^{b} | thioridazine | | zimelidine | | ketoconazole | | loratadine | | pentamidine | | acetaminophen | |
| real-time | kiaa1001 | 5.79 | | 2.24 | * | 2.24 | ** | 1.42 | | 1.82 | ** | 1.13 | |
| quantitative | asah1 | 1.36 | | 2.91 | | 1.47 | | 1.23 | | 0.93 | | 1.18 | |
| PCR | mgc4171 | 1.32 | | 1.12 | | 1.53 | * | 1.57 | | 0.73 | | 0.75 | |
| | lss | 2.03 | | 1.05 | | 4.33 | ** | 1.54 | | 0.52 | | 0.91 | |
| | nr0b2 | 0.77 | | 1.33 | | 2.36 | ** | 1.63 | | 1.83 | ** | 0.69 | |
| | fabp1 | 5.07 | * | 1.42 | | 5.30 | ** | 1.58 | | 0.15 | * | 1.11 | |
| | hpn | 2.85 | | 1.74 | ** | 2.71 | ** | 1.52 | * | 2.50 | | 1.10 | |
| | serpina3 | 2.46 | ** | 1.95 | | 2.37 | * | 1.83 | * | 2.12 | * | 0.99 | |
| | depp | 2.02 | ** | 2.67 | ** | 2.29 | ** | 0.79 | | 2.88 | ** | 0.94 | |
| | flj22362 | 1.57 | | 1.71 | | 1.95 | | 1.12 | | 2.49 | ** | 1.02 | |
| | slc2a3 | 0.38 | | 0.70 | | 0.48 | * | 0.46 | * | 0.50 | * | 1.50 | |
| | tagln | 0.46 | ** | 0.59 | * | 0.71 | | 0.44 | ** | 0.68 | | 0.93 | ** |

| electro- | | frequency of appearance of myelin-like structure | | | | | | | | |
|---|---|---|---|---|---|---|---|---|---|---|
| microscopic | | − | | | | | | | | |
| test | | expression variation rate | | | | | | | | |
| | gene | clarithromycin | disopyramide | erythromycin | flecainide | | haloperidol | | levofloxacin | |
| real-time | kiaa1001 | 1.48 | 0.94 | 0.78 | 2.10 | * | 1.32 | | 0.70 | |
| quantitative | asah1 | 1.55 | 1.85 | 0.90 | 2.42 | | 0.77 | | 1.10 | |
| PCR | mgc4171 | 1.18 | 1.21 | 0.86 | 1.57 | | 1.24 | | 0.79 | |
| | lss | 0.65 | 1.14 | 0.87 | 1.22 | | 1.52 | | 0.75 | |
| | nr0b2 | 0.78 | 0.63 | 0.90 | 1.02 | | 1.49 | * | 0.79 | |
| | fabp1 | 0.91 | 1.02 | 0.73 | 1.50 | | 1.50 | * | 0.88 | |
| | hpn | 0.96 | 1.12 | 0.87 | 1.26 | | 1.43 | | 0.83 | |
| | serpina3 | 1.39 | 0.99 | 0.85 | 1.60 | | 1.31 | | 0.92 | |
| | depp | 0.93 | 0.90 | 0.71 | 1.73 | | 1.38 | | 0.83 | * |
| | flj22362 | 1.00 | 0.98 | 0.92 | 1.65 | | 0.93 | | 0.83 | |
| | slc2a3 | 0.86 | 1.03 | 0.73 | 0.46 | | 0.44 | * | 1.07 | |
| | tagln | 0.86 | 0.99 | 0.85 | 0.86 | | 0.52 | * | 0.90 | |

| electro- | | frequency of appearance of myelin-like structure | | | | | | | | | | |
|---|---|---|---|---|---|---|---|---|---|---|---|---|
| microscopic | | − | | | | | | | | | | |
| test | | expression variation rate | | | | | | | | | | |
| | gene | ofloxacin | | procainamide | | quinidine | | sotalol | sulfamethoxazole | | sumatriptan | |
| real-time | kiaa1001 | 0.86 | | 1.78 | | 1.27 | | 0.84 | 0.84 | | 1.34 | |
| quantitative | asah1 | 1.17 | | 0.86 | | 1.43 | | 1.39 | 1.26 | | 1.35 | |
| PCR | mgc4171 | 0.77 | | 1.08 | | 1.20 | | 0.72 | 0.78 | | 0.99 | |
| | lss | 0.78 | | 0.90 | | 1.30 | | 0.88 | 0.82 | | 1.23 | * |
| | nr0b2 | 0.81 | | 0.74 | | 1.03 | | 0.63 | 0.82 | | 0.63 | |
| | fabp1 | 0.75 | | 1.00 | | 1.38 | | 0.98 | 0.85 | | 1.01 | |
| | hpn | 0.72 | | 1.08 | | 1.21 | | 0.79 | 0.89 | | 1.16 | |
| | serpina3 | 0.82 | | 0.96 | | 1.22 | | 0.78 | 0.98 | | 0.95 | |
| | depp | 0.79 | * | 0.98 | | 1.06 | | 0.80 | 0.70 | * | 1.05 | |
| | flj22362 | 0.68 | * | 0.73 | * | 1.26 | | 0.85 | 0.87 | | 1.26 | |
| | slc2a3 | 0.94 | | 0.88 | | 0.37 | ** | 1.53 | 1.11 | | 0.99 | |
| | tagln | 0.74 | ** | 0.85 | | 0.63 | * | 0.80 | 0.99 | | 1.06 | |

^{a}see Example 1 (Reference Example)
^{b}abbreviation of PLsis marker gene
^{c}relative value (average value) of gene expression amount of compound addition group when gene expression amount (average value) of control group is 1.0
*, ** significance from control group (t-test) *, p < 0.05; **, p < 0.01

An average variation rate of expression of 12 genes (each gene was considered to have the same weight) by each test compound was calculated. The correlation between the average variation rate and the emergence of a myelin-like structure was examined and the results are shown in FIG. 6. Those having a higher average variation rate tended to show a high frequency of emergence of a myelin-like structure, and a good correlation was found between them. When the judgment standard value for the evaluation of the presence or absence of a PLsis induction potential was an average variation rate of 1.5, 17 PLsis-inducing compounds all showed an average variation rate of not less than 1.5. In contrast, 12 compounds out of 13 PLsis non-inducing compounds showed an average variation rate of less than 1.5. Thus, the presence or absence of a PLsis induction potential could be correctly evaluated with a 30 compounds-29 compounds (about 97%) probability of correctness of evaluation.

### Example 3

Confirmation of Reliability of Evaluation System

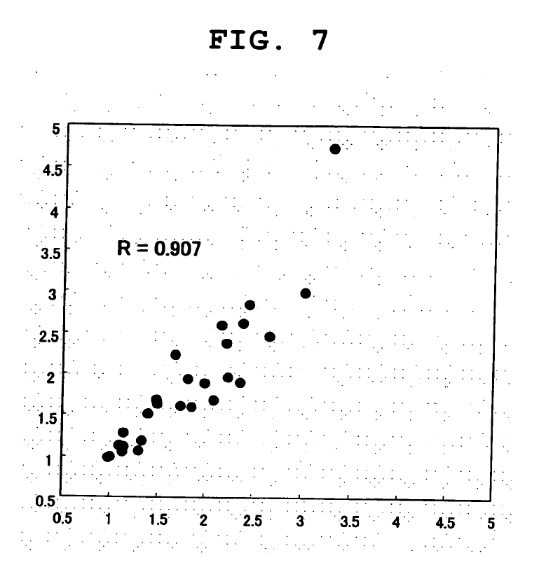

In the same manner as in Example 1 (Reference Example), HepG2 cells were exposed to 26 kinds of compounds unknown as to the presence or absence of a PLsis induction potential for 24 hr each, and in the same manner as in Example 2, expression variation rate of 12 PLsis marker genes was determined and an average variation rate (each gene was considered to have the same weight) was calculated. The same test was performed twice. The average variation rate of the first test is shown in the x coordinate, the average variation rate of the second test is shown in the y coordinate and the results of each test compound are plotted in FIG. 7. Comparison of the two test results revealed good reproducibility (R=0.907). The presence or absence of emergence of a myelin-like structure upon exposure of HepG2 cells to these 26 compounds for 72 hr was separately detected according to the method of Reference Example, and the relationship with an average variation rate was examined. As a result, PLsis negative compounds were distributed in the area of an average variation rate of less than 1.5 in the graph and PLsis positive compounds were distributed in the area of an average variation rate of not less than 1.5, thus showing an extremely good prediction hitting ratio.

### INDUSTRIAL APPLICABILITY

The PLsis prediction method of the present invention characteristically detects expression variation of a PLsis marker gene upon exposure of mammalian cells to a compound. Therefore, it affords an advantageous effect of possible rapid and convenient examination of many compounds, as compared to conventional in vivo toxicity test and evaluation methods using enzyme activity, intracellular accumulation of phospholipid and the like as indices.

In addition, the toxicity prediction method of the present invention affords an advantageous effect of possible more accurate prediction of the presence or absence of toxicity, as compared to conventional evaluation methods using, as an index, an average expression variation rate of gene groups showing common variation of expression in correlation with the toxicity expression.

The prediction method of a PLsis induction potential of a compound according to the present invention shows low probability of false-positive and false-negative, and is an in vitro evaluation system of a PLsis induction potential, which is superior in reliability. In addition, since the method can treat many samples more rapidly and more conveniently than conventional methods, it is particularly useful for the toxicity evaluation of drug candidate compounds in the initial stages of drug discovery.

Sequence Listing Free Text

SEQ ID NO:25: oligonucleotide designed to function as a primer for amplifying kiaa1001 gene transcription product.

SEQ ID NO:26: oligonucleotide designed to function as a primer for amplifying kiaa1001 gene transcription product.

SEQ ID NO:27: oligonucleotide designed to function as TaqMan probe for detecting kiaa1001 gene transcription product.

SEQ ID NO:28: oligonucleotide designed to function as a primer for amplifying asah1 gene transcription product.

SEQ ID NO:29: oligonucleotide designed to function as a primer for amplifying asah1 gene transcription product.

SEQ ID NO:30: oligonucleotide designed to function as TaqMan probe for detecting asah1 gene transcription product.

SEQ ID NO:31: oligonucleotide designed to function as a primer for amplifying mgc4171 gene transcription product.

SEQ ID NO:32: oligonucleotide designed to function as a primer for amplifying mgc4171 gene transcription product.

SEQ ID NO:33: oligonucleotide designed to function as TaqMan probe for detecting mgc4171 gene transcription product.

SEQ ID NO:34: oligonucleotide designed to function as a primer for amplifying lss gene transcription product.

SEQ ID NO:35: oligonucleotide designed to function as a primer for amplifying lss gene transcription product.

SEQ ID NO:36: oligonucleotide designed to function as TaqMan probe for detecting lss gene transcription product.

SEQ ID NO:37: oligonucleotide designed to function as a primer for amplifying nr0b2 gene transcription product.

SEQ ID NO:38: oligonucleotide designed to function as a primer for amplifying nr0b2 gene transcription product.

SEQ ID NO:39: oligonucleotide designed to function as TaqMan probe for detecting nr0b2 gene transcription product.

SEQ ID NO:40: oligonucleotide designed to function as a primer for amplifying fabp1 gene transcription product.

SEQ ID NO:41: oligonucleotide designed to function as a primer for amplifying fabp1 gene transcription product.

SEQ ID NO:42: oligonucleotide designed to function as TaqMan probe for detecting fabp1 gene transcription product.

SEQ ID NO:43: oligonucleotide designed to function as a primer for amplifying hpn gene transcription product.

SEQ ID NO:44: oligonucleotide designed to function as a primer for amplifying hpn gene transcription product.

SEQ ID NO:45: oligonucleotide designed to function as TaqMan probe for detecting hpn gene transcription product.

SEQ ID NO:46: oligonucleotide designed to function as a primer for amplifying serpina3 gene transcription product.

SEQ ID NO:47: oligonucleotide designed to function as a primer for amplifying serpina3 gene transcription product.

SEQ ID NO:48: oligonucleotide designed to function as TaqMan probe for detecting serpina3 gene transcription product.

SEQ ID NO:49: oligonucleotide designed to function as a primer for amplifying depp gene transcription product.

SEQ ID NO:50: oligonucleotide designed to function as a primer for amplifying depp gene transcription product.

SEQ ID NO:51: oligonucleotide designed to function as TaqMan probe for detecting depp gene transcription product.

SEQ ID NO:52: oligonucleotide designed to function as a primer for amplifying flj22362 gene transcription product.

SEQ ID NO:53: oligonucleotide designed to function as a primer for amplifying flj22362 gene transcription product.

SEQ ID NO:54: oligonucleotide designed to function as TaqMan probe for detecting flj22362 gene transcription product.

SEQ ID NO:55: oligonucleotide designed to function as a primer for amplifying slc2a3 gene transcription product.

SEQ ID NO:56: oligonucleotide designed to function as a primer for amplifying slc2a3 gene transcription product.

SEQ ID NO:57: oligonucleotide designed to function as TaqMan probe for detecting slc2a3 gene transcription product.

SEQ ID NO:58: oligonucleotide designed to function as a primer for amplifying tagln gene transcription product.

SEQ ID NO:59: oligonucleotide designed to function as a primer for amplifying tagln gene transcription product.

SEQ ID NO:60: oligonucleotide designed to function as TaqMan probe for detecting tagln gene transcription product.

This application is based on a patent application No. 2003-397551 filed in Japan (filing date: Nov. 27, 2003), the contents of which are incorporated in full herein by this reference.

1. A reagent for predicting a phospholipidosis induction potential of a compound, which comprises a nucleic acid capable of hybridizing to a nucleic acid having a base sequence shown by any of SEQ ID NOs:1, 3, 5, 7, 9, 11, 13, 15, 17, 19, 21 and 23 under high stringent conditions and/or a nucleic acid capable of hybridizing to a nucleic acid having a base sequence complementary to the base sequence under high stringent conditions.

2. A kit for predicting a phospholipidosis induction potential of a compound, which comprises one or more reagents containing a nucleic acid capable of hybridizing to a transcription product of a gene showing varying expression in correlation with expression of phospholipidosis under high stringent conditions and/or a nucleic acid capable of hybridizing to a nucleic acid having a base sequence complementary to the transcription product under high stringent conditions, wherein, when two or more reagents are contained, each reagent can detect expression of different genes.

3. The kit of claim 2, wherein at least one reagent comprises a nucleic acid capable of hybridizing to a nucleic acid having a base sequence shown by any of SEQ ID NOs:1, 3, 5, 7, 9, 11, 13, 15, 17, 19, 21 and 23 under high stringent conditions and/or a nucleic acid capable of hybridizing to a nucleic acid having a base sequence complementary to the base sequence under high stringent conditions.

4. The kit of claim 2, wherein a prediction hitting ratio of the phospholipidosis induction potential is not less than about 70% when a mammalian cell is exposed to a test compound, using an average variation rate of expression of a nucleic acid, to which the nucleic acid contained in each reagent is capable of hybridizing, in said cell as an index.

5. A method for predicting a phospholipidosis induction potential of a compound, which comprises detecting expression variation of one or more genes showing expression variation in correlation with phospholipidosis expression, in a sample containing a mammalian cell exposed to the compound or a sample taken from a mammal administered with the compound.

6. The method of claim 5, wherein at least one gene has the same or substantially the same base sequence as the base sequence shown by any of SEQ ID NOs:1, 3, 5, 7, 9, 11, 13, 15, 17, 19, 21 and 23.

7. A method for determining the standard for the judgment of the presence or absence of a phospholipidosis induction potential of a compound, which comprises
(1) detecting expression variation of one or more genes showing expression variation in correlation with phospholipidosis expression, in samples containing a mammalian cell exposed to each of two or more known phospholipidosis-inducing compounds and two or more known phospholipidosis non-inducing compounds or samples taken from mammals administered with each of said compounds, and
(2) using, as a standard value, an average variation rate capable of correctly judging the presence or absence of a phospholipidosis induction potential of the above-mentioned compounds by not less than about 70% based on the relationship between an average expression variation rate of the genes and the phospholipidosis induction potential.

8. The method of claim 7, wherein at least one gene has the same or substantially the same base sequence as the base sequence shown by any of SEQ ID NOs:1, 3, 5, 7, 9, 11, 13, 15, 17, 19, 21 and 23.

9. The method of claim 7, further comprising examining validity of the standard value using other known phospholipidosis inducing compound and known phospholipidosis non-inducing compound.

10. The method of claim 5, comprising comparing the average variation rate of gene expression with the standard value obtained by the method of claim 7 or 9.

11. A method for predicting the toxicity of a compound, which comprises,
(1) detecting expression variation of one or more genes showing expression variation in correlation with toxicity expression, in a sample containing a mammalian cell exposed to the compound or a sample taken from a mammal administered with the compound, and
(2) judging the presence or absence of toxicity of the compound with an average variation rate of the gene expression as an index.
